(19) United States
(12) Patent Application Publication
(10) Pub. No.: US 2025/0228534 A1
(43) Pub. Date: Jul. 17, 2025

(54) MINIMALLY INVASIVE INSERTION SYSTEM FOR NEURAL INTERFACES

(71) Applicant: PRECISION NEUROSCIENCE CORPORATION, New York, NY (US)

(72) Inventors: Adam POOLE, New York, NY (US); Morgan LaMarca, New York, NY (US); Benjamin I. RAPOPORT, New York, NY (US)

(73) Assignee: PRECISION NEUROSCIENCE CORPORATION, New York, NY (US)

(21) Appl. No.: 18/434,008

(22) Filed: Feb. 6, 2024

Related U.S. Application Data
(60) Provisional application No. 63/621,353, filed on Jan. 16, 2024.

(51) Int. Cl. A61B 17/00; A61B 17/14; A61B 17/15; A61B 17/16; A61B 17/17

(52) U.S. Cl. CPC A61B 17/00234; A61B 17/147; A61B 17/15; A61B 17/1695; A61B 17/17; A61B 2017/00238; A61B 2017/00853

(57) ABSTRACT

A surgical system and method for use in implanting a neural device in a minimally invasive manner. The surgical system can include an adjustable cranial guide and various surgical tools that are dimensioned for creating a minimally invasive osteotomy and working within the subdural space.

### CROSS-REFERENCE TO PRIOR APPLICATIONS

This application claims priority to U.S. Provisional Application No. 63/621,353, filed Jan. 16, 2024, the entirety of which is incorporated herein by reference.

### BACKGROUND

Brain-computer interfaces have shown promise as systems for restoring, replacing, and augmenting lost or impaired neurological function in a variety of contexts, including paralysis from stroke and spinal cord injury, blindness, and some forms of cognitive impairment. Multiple innovations over the past several decades have contributed to the potential of these neural interfaces, including advances in the areas of applied neuroscience and multichannel electrophysiology, mathematical and computational approaches to neural decoding, power-efficient custom electronics and the development of application-specific integrated circuits, as well as materials science and device packaging. Nevertheless, the practical impact of such systems remains limited, with only a small number of patients worldwide having received highly customized interfaces through clinical trials.

High bandwidth brain-computer interfaces are being developed to enable the bidirectional communication between the nervous system and external computer systems in order to assist, augment, or replace neurological function lost to disease or injury. A necessary capability of any brain-computer interface is the ability to accurately decode electrophysiologic signals recorded from individual neurons, or populations of neurons, and correlate such activity with one or more sensory stimuli or intended motor response. For example, such a system may record activity from the primary motor cortex in an animal or a paralyzed human patient and attempt to predict the actual or intended movement in a specific body part; or the system may record activity from the visual cortex and attempt to predict both the location and nature of the stimuli present in the patient's visual field.

Furthermore, brain-penetrating microelectrode arrays have facilitated high-spatial-resolution recordings for brain-computer interfaces, but at the cost of invasiveness and tissue damage that scale with the number of implanted electrodes. In some applications, softer electrodes have been used in brain-penetrating microelectrode arrays; however, it is not yet clear whether such approaches offer a substantially different tradeoff as compared to conventional brain-penetrating electrodes. For this reason, non-penetrating cortical surface microelectrodes represent a potentially attractive alternative and form the basis of the system described here. In practice, electrocorticography (ECOG) has already facilitated capture of high quality signals for effective use in brain-computer interfaces in several applications, including motor and speech neural prostheses. Higher-spatial-resolution micro-electrocorticography (uECoG) therefore represents a promising combination of minimal invasiveness and improved signal quality. Therefore, it would be highly beneficial for neural devices to make use of non-penetrating cortical interfaces.

One omnipresent challenge in the brain-computer interface field is implanting the neural devices in subjects in a manner that causes the least amount of surgical impact on the subjects. This is especially challenging for neural devices because such devices necessarily require accessing the patient's brain, which is traditionally a highly invasive procedure. Therefore, it would be highly beneficial for surgical techniques and tools to be developed that allowed for the subdural space to be accessed in a minimally invasive manner.

### SUMMARY

The present disclosure is directed to systems and methods for surgically inserting and/or implanting brain-computer interfaces and related medical devices in a subject.

In some embodiments, there is provided a minimally invasive surgical system for implanting a neural interface. The minimally invasive surgical system can include one or more of any of the components described below, including an incision template, a cranial guide system, a micro Kerrison rongeur, a stylet, a shim, a size gauge, a visualization system, a micro pick, a neural interface anchor, or any combination thereof.

In one embodiment, there is provided a cranial guide for surgically preparing a subject for an implantable neural device, the cranial guide comprising: a saw slot for receiving an oscillating saw blade, the saw slot oriented to form an angular slit in the subject oriented from about 45° to about 65°; and a drill aperture for receiving a drill bit therethrough, the drill aperture coextensive with the saw slot.

In one embodiment, there is provided a method for surgically preparing a subject for an implantable neural device, the method comprising: placing an incision template on a head of the subject, the incision template comprising a first portion and a second portion arranged orthogonally with respect to the first portion, the first portion defining a slit configured to receive a cutting instrument therethrough, wherein a first length of the slit corresponds to a second length of an angular slit to be formed in the subject; using the incision template, making an incision in a scalp of the subject with the cutting instrument; placing a cranial guide on the scalp of the subject at the incision, the cranial guide comprising a saw slot for receiving an oscillating saw blade and a drill aperture for receiving a drill bit therethrough, wherein the drill aperture is coextensive with the saw slot; using the drill aperture of the cranial guide, drilling a pilot hole through the scalp and into a skull of the subject; and using the saw slot, cutting the angular slit through the scalp and into the skull of the subject, wherein oriented from about 45° to about 65°.

In some embodiments, the cranial guide system further comprises leveling pins to adjust a relative position or orientation of the cranial guide relative to the subject.

In some embodiments, the cranial guide system further comprises a friction-reducing coating disposed on an inner surface of the saw slot.

In some embodiments, the coating comprises at least one of polytetrafluoroethylene, Xylan® 8110, Eclipse®, plasma polymerized coating, diamond-like carbon (DLC) coating, or lubricious silicone coating.

In some embodiments, the drill aperture is disposed at a midpoint of the saw slot.

In some embodiments, a thickness of the saw slot is from about 0.4 mm to about 0.8 mm.

In some embodiments, a thickness of the saw slot is from about 0.8 mm to about 1 mm. In some embodiments, a width of the drill aperture is from about 0.5 mm to about 3 mm.

### FIGURES

FIG. 1 depicts a block diagram of a secure neural device data transfer system, in accordance with an embodiment of the present disclosure.

FIG. 2 depicts a diagram of a neural device, in accordance with an embodiment of the present disclosure.

FIG. 3 depicts a diagram of a thin-film, microelectrode array neural device and implantation method, in accordance with an embodiment of the present disclosure.

FIG. 4 depicts a flow diagram of a process for preparing a subject for implantation of a neural device in a minimally invasive manner, in accordance with an embodiment of the present disclosure.

FIG. 5A depicts an osteotomy created using the minimally invasive process described in connection with FIG. 4, in accordance with an embodiment of the present disclosure.

FIG. 5B depicts a neural device being inserted through the osteotomy shown in FIG. 5A, in accordance with an embodiment of the present disclosure.

FIG. 5C depicts the deployed neural device shown in FIG. 5B, in accordance with an embodiment of the present disclosure.

FIG. 6A depicts a diagram showing the placement of the skin incision and cranial guide for the planned trajectory between the target location for the neural device against the cortical surface and the entry point of the patient's skull, in accordance with an embodiment of the present disclosure.

FIG. 6B depicts an angular slit and pilot hole formed in a skull, in accordance with an embodiment of the present disclosure.

FIG. 7 depicts an incision template, in accordance with an embodiment of the present disclosure.

FIG. 8A depicts a first perspective view of the mount of a cranial guide system, in accordance with an embodiment of the present disclosure.

FIG. 8B depicts a second perspective view of the mount of a cranial guide system, in accordance with an embodiment of the present disclosure.

FIG. 8C depicts a third perspective view of the mount of a cranial guide system, in accordance with an embodiment of the present disclosure.

FIG. 8D a perspective view of an alternative embodiment of the mount of a cranial guide system, in accordance with an embodiment of the present disclosure.

FIG. 8E depicts a phantom view of a guide portion affixed to a mount, in accordance with an embodiment of the present disclosure.

FIG. 8F depicts a perspective view of the cranial guide system secured to a subject's skull with adjustable leveling pins, in accordance with an embodiment of the present disclosure.

FIG. 8G depicts another perspective view of the cranial guide system to secured to a subject's skull, in accordance with an embodiment of the present disclosure.

FIG. 8H depicts an overhead view of the cranial guide system to secured to a subject's skull, in accordance with an embodiment of the present disclosure.

FIG. 8I depicts another perspective view of the cranial guide system to secured to a subject's skull, in accordance with an embodiment of the present disclosure.

FIG. 8J depicts various views of the cranial guide system with illustrative dimensions, in accordance with an embodiment of the present disclosure.

FIG. 8K depicts a perspective view of a screw leveling pin with illustrative dimensions, in accordance with an embodiment of the present disclosure.

FIG. 8L depicts another perspective view of the screw leveling pin depicted in FIG. 8K, in accordance with an embodiment of the present disclosure.

FIG. 8M depicts a phantom view of the threaded aperture in the cranial guide system mount configured to receive a screw leveling pin, in accordance with an embodiment of the present disclosure.

FIG. 8N depicts a phantom view of the threaded aperture in the cranial guide system mount with a screw leveling pin engaged therewith, in accordance with an embodiment of the present disclosure.

FIG. 8O depicts different embodiments of the guide portion configured for different surgical trajectories, in accordance with embodiments of the present disclosure.

FIG. 9A depicts a first view of a cranial guide system being used to drill the pilot hole, in accordance with an embodiment of the present disclosure.

FIG. 9B depicts a second view of a cranial guide system being used to drill the pilot hole, in accordance with an embodiment of the present disclosure.

FIG. 9C depicts a third view of a cranial guide system being used to drill the pilot hole, in accordance with an embodiment of the present disclosure.

FIG. 9D depicts the drill bit for drilling the pilot hole having a friction-reducing coating, in accordance with an embodiment of the present disclosure.

FIG. 9E depicts the drill stop for drilling the pilot hole having a friction-reducing coating, in accordance with an embodiment of the present disclosure.

FIG. 9F depicts a drill engaged with a drill stop set at a first depth, in accordance with an embodiment of the present disclosure.

FIG. 9G depicts the drill stop being moved from the first depth to a second depth, in accordance with an embodiment of the present disclosure.

FIG. 9H depicts a drill engaged with a drill stop set at a second depth, in accordance with an embodiment of the present disclosure.

FIG. 9I depicts an exploded view of the adjustable drill stop, in accordance with an embodiment of the present disclosure.

FIG. 9J depicts a phantom view of the portions of the drill stop engaged with each other to set a desired depth, in accordance with an embodiment of the present disclosure.

FIG. 10A depicts a first perspective view of a guide portion of a cranial guide system, in accordance with an embodiment of the present disclosure.

FIG. 10B depicts a second perspective view of a guide portion of a cranial guide system, in accordance with an embodiment of the present disclosure.

FIG. 10C depicts a third perspective view of a guide portion of a cranial guide system, in accordance with an embodiment of the present disclosure.

FIG. 10D depicts a fourth perspective view of a guide portion of a cranial guide system, in accordance with an embodiment of the present disclosure.

FIG. 10E depicts a sectional view of a guide portion having a double width saw slot, in accordance with an embodiment of the present disclosure.

FIG. 10F depicts a sectional view of a guide portion having a single width saw slot, in accordance with an embodiment of the present disclosure.

FIG. 11A depicts a first exploded view of a cranial guide system, in accordance with an embodiment of the present disclosure.

FIG. 11B depicts a second exploded view of a cranial guide system, in accordance with an embodiment of the present disclosure.

FIG. 11C depicts a perspective view of a cranial guide system in a partially assembled configuration, in accordance with an embodiment of the present disclosure.

FIG. 11D depicts a perspective view of a cranial guide system in a fully assembled configuration, in accordance with an embodiment of the present disclosure.

FIG. 12A depicts a first perspective view of a guide portion wherein the saw slot includes a coating, in accordance with an embodiment of the present disclosure.

FIG. 12B depicts a second perspective view of a guide portion wherein the saw slot includes a coating, in accordance with an embodiment of the present disclosure.

FIG. 13A depicts a perspective view of a saw blade unloaded from the cranial guide system, in accordance with an embodiment of the present disclosure.

FIG. 13B depicts a perspective view of a saw blade partially inserted into the cranial guide system, in accordance with an embodiment of the present disclosure.

FIG. 13C depicts a perspective view of a saw blade fully inserted into the cranial guide system, in accordance with an embodiment of the present disclosure.

FIG. 13D depicts a perspective view of a saw blade at its stop position within the cranial guide system, in accordance with an embodiment of the present disclosure.

FIG. 14A depicts an elevational view of a saw blade for use with the cranial guide system, in accordance with an embodiment of the present disclosure.

FIG. 14B depicts a first perspective view of the saw blade depicted in FIG. 14A, in accordance with an embodiment of the present disclosure.

FIG. 14C depicts a second perspective view of the saw blade depicted in FIG. 14A, in accordance with an embodiment of the present disclosure.

FIG. 15A depicts a first perspective view of a saw blade secured to a saw handpiece, in accordance with an embodiment of the present disclosure.

FIG. 15B depicts a second perspective view of a saw blade secured to a saw handpiece, in accordance with an embodiment of the present disclosure.

FIG. 15C depicts a third perspective view of a saw blade secured to a saw handpiece, in accordance with an embodiment of the present disclosure.

FIG. 15D depicts a fourth perspective view of a saw blade secured to a saw handpiece, in accordance with an embodiment of the present disclosure.

FIG. 15E depicts a perspective view of an adjustable stop attachment for a sagittal saw, in accordance with an embodiment of the present disclosure.

FIG. 15F depicts another perspective view of the adjustable stop attachment showing the clamping latch, in accordance with an embodiment of the present disclosure.

FIG. 15G depicts a top-down view of the adjustable stop attachment with the cam in the unlocked position, in accordance with an embodiment of the present disclosure.

FIG. 15H depicts a top-down view of the adjustable stop attachment with the cam in the locked position, in accordance with an embodiment of the present disclosure.

FIG. 15I depicts a first side view of the adjustable stop attachment, in accordance with an embodiment of the present disclosure.

FIG. 15J depicts a second side view of the adjustable stop attachment, in accordance with an embodiment of the present disclosure.

FIG. 16 depicts a perspective view of an irrigation device used with the cranial guide system, in accordance with an embodiment of the present disclosure.

FIG. 17A depicts a perspective view of a micro Kerrison rongeur in an open configuration, in accordance with an embodiment of the present disclosure.

FIG. 17B depicts a perspective view of the micro Kerrison rongeur of FIG. 17A in a closed configuration, in accordance with an embodiment of the present disclosure.

FIG. 18A depicts a view of a stylet, a shim, and a size gauge, in accordance with an embodiment of the present disclosure.

FIG. 18B depicts a view of a combination stylet and space tool, a stylet, and a size gauge, in accordance with an embodiment of the present disclosure.

FIG. 18C depicts a partially coated deployment stylet, in accordance with an embodiment of the present disclosure.

FIG. 19A depicts the stylet shown in FIG. 18A being used to deploy a neural device within the subdural space at the cortical surface, in accordance with an embodiment of the present disclosure.

FIG. 19B depicts another embodiment of a neural device being deployed within the subdural space at the cortical surface, in accordance with an embodiment of the present disclosure.

FIG. 20 depicts a diagram of an endoscopic surgical setup, in accordance with an embodiment of the present disclosure.

FIG. 21A depicts a perspective view of a tissue pick, in accordance with an embodiment of the present disclosure.

FIG. 21B depicts a first elevational view of a tissue pick, in accordance with an embodiment of the present disclosure.

FIG. 21C depicts a second elevational view of the tissue pick depicted in FIG. 21B, in accordance with an embodiment of the present disclosure.

FIG. 21D depicts a first elevational view of the distal end of the tissue pick depicted in FIG. 21B, in accordance with an embodiment of the present disclosure.

FIG. 21E depicts a second elevational view of the distal end of the tissue pick depicted in FIG. 21B, in accordance with an embodiment of the present disclosure.

FIG. 21F depicts a pair of the tissue picks depicted in FIG. 21B, in accordance with an embodiment of the present disclosure.

FIG. 21G depicts a first view of the tissue pick depicted in FIGS. 21B-21F being used to lift dura tissue, in accordance with an embodiment of the present disclosure.

FIG. 21H depicts a second view of the tissue pick depicted in FIGS. 21B-21F being used to lift dura tissue, in accordance with an embodiment of the present disclosure.

FIG. 22 depicts views of a pick knife, in accordance with an embodiment of the present disclosure.

FIG. 23A depicts a first perspective view of a hook knife, in accordance with an embodiment of the present disclosure.

FIG. 23B depicts a second perspective view of a hook knife, in accordance with an embodiment of the present disclosure.

FIG. 23C depicts an elevational view of an alternative embodiment of a hook knife, in accordance with an embodiment of the present disclosure.

FIG. 24A depicts a surgical setup for an endoscope and various surgical tools, in accordance with an embodiment of the present disclosure.

FIG. 24B depicts another surgical setup for an endoscope and various surgical tools, in accordance with an embodiment of the present disclosure.

FIG. 25A depicts anchors securing electrode array cables in place, in accordance with an embodiment of the present disclosure.

FIG. 25B depicts a first side of an electrode array anchor, in accordance with an embodiment of the present disclosure.

FIG. 25C depicts a second side of an electrode array anchor, in accordance with an embodiment of the present disclosure.

FIG. 25D depicts an alternative embodiment of an electrode array anchor, in accordance with an embodiment of the present disclosure.

### DETAILED DESCRIPTION

The present disclosure is generally directed to surgical systems and methods for implanting neural devices, particularly neural devices having non-penetrating electrodes. In particular, the present is disclosure to surgical techniques and tools for implanting neural devices in a minimally invasive manner.

Disclosed herein is a delivery system for high-bandwidth neural interfaces for the brain that implements a minimally invasive approach, which reduces damage to the subject's brain and surrounding tissues, while retaining compatibility with existing endoscopic, stereotactic, image guided, and robot-assisted surgical techniques. Embodiments may include the surgical approach and mechanical system for implantation of biocompatible devices that can be implanted into the brain to form a brain-computer interface. Embodiments of the present disclosure include the surgical technique for implantation of high-bandwidth neural interfaces. In some embodiments, a slit-like incision [osteotomy] may be made in the skull tangentially to the cortical surface using an oscillating bone saw or other tool. In some embodiments, novel microsurgical instruments may be used to safely incise the dura mater through the osteotomy without damaging the brain surface.

Embodiments of the present disclosure include the surgical delivery system for implanting high-bandwidth neural interfaces. In some embodiments, the surgical delivery system can be used to make one or more slit-like incisions through the scalp, skull, and dura matter that are oriented tangentially to the surface of the brain. In some embodiments, the surgical delivery system may deploy the neural interface in the subdural space onto the surface of the cortex through the mechanical guidance of a semi-flexible stylet.

In some embodiments, the deployment system may implement precision tooling and fixturing that is compatible with existing stereotactic neurosurgical systems, such as the stereotactic system from Leksell® or the head frame from Mayfield®, and/or existing neurosurgical navigation and robotic systems, such as the ROSA ONE® robot from Zimmer Biomet®, StealthStation™ from Medtronic®, or the neurosurgical system from Brainlab®. Embodiments of the present disclosure may include methods of validation for the deployment and positional accuracy of the microelectrode array. The delivery system may include the implementation of a small diameter endoscope coaxial to the slit incision for visual feedback and confirmation. In some embodiments, one or more components of the surgical system that are introduced into the subdural space can be individually trackable and/or uniquely identifiable through electromagnetic detection and/or radiopaque markers for fluoroscopy, computed tomography (CT), or other imaging modalities. For example, electrode array placement can be validated via endoscopy to directly visualize the placement of the electrode array(s) on the cortical surface. As another example, fluoroscopy can be used to track radiopaque features on the electrode array to validate array placement. As yet another example, the subject's electrophysiologic response to evoked potentials can be used to validate array placement. Additionally, or alternatively, other forms of target location validation may be implemented through use of real time electrophysiology techniques.

### Neural Device Systems

Conventional neural devices typically include electrode arrays that penetrate a subject's brain in order to sense and/or stimulate the brain. However, the present disclosure is directed to the use of non-penetrating neural devices, i.e., neural devices having electrode arrays that do not penetrate the cortical surface. Such non-penetrating neural devices are minimally invasive and minimize the amount of impact on the subject's cortical tissue. Neural devices can sense and record brain activity, receive instructions for stimulating the subject's brain, and otherwise interact with a subject's brain as generally described herein.

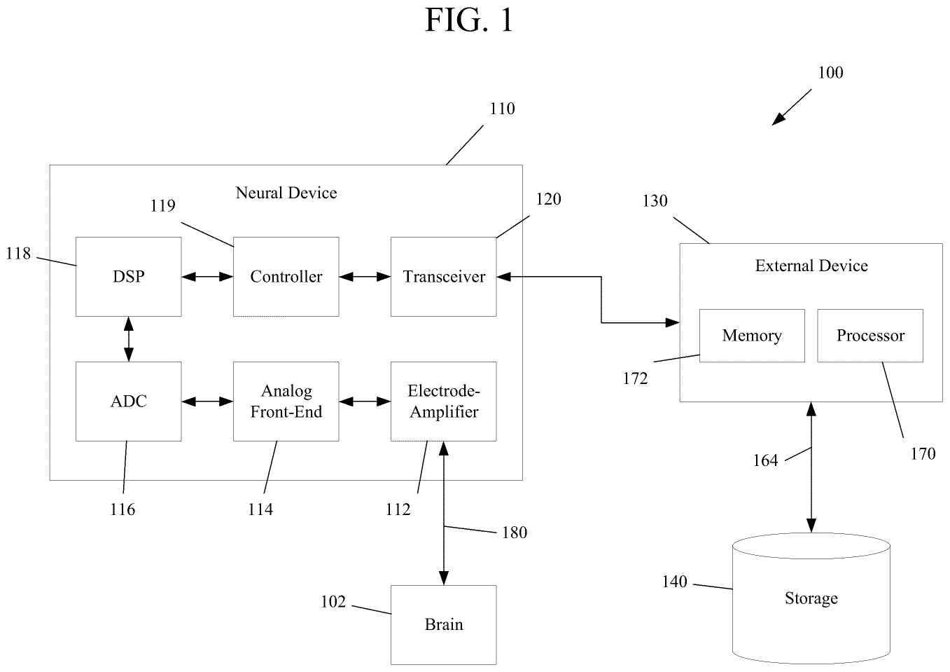

Referring now to FIG. 1, there is shown a diagram of an illustrative system 100 including a neural device 110 that is communicatively coupled to an external device 130. The external device 130 can include any device to which the neural device 110 can be communicatively coupled, such as a computer system or mobile device (e.g., a tablet, a smartphone, a laptop, a desktop, a secure server, a smartwatch, a head-mounted virtual reality device, a head-mounted augmented reality device, or a smart inductive charger device). The external device 130 can include a processor 140 and a memory 142. In some embodiments, the computer system or mobile device can include a server or a cloud-based computing system. In some embodiments, the external device 130 can further include or be communicatively coupled to storage 140. In one embodiment, the storage 140 can include a database stored on the external device 130. In another embodiment, the storage 140 can include a cloud computing system (e.g., Amazon Web Services or Azure). The external device 130 can include a processor 170 and a memory 172. In some embodiments, the external device 130 can include a server or a cloud-based computing system. In some embodiments, the external device 130 can further include or be communicatively coupled to storage 140. In one embodiment, the storage 140 can include a database stored on the external device 130. In another embodiment, the storage 140 can include a cloud computing system (e.g., Amazon Web Services or Azure).

In some embodiments, the electrode array 180 of the neural device 110 can have electrodes that are sufficiently small and spaced at sufficiently small distances in order to define a high-density electrode array 180 that can, accordingly, capture high resolution electrocortical data. Such high-resolution data can be used to resolve electrographic features that can otherwise not be identified using lower resolution electrode arrays. In some embodiments, the electrodes of the electrode array 180 can be from about 10 μm to about 500 μm in width. In one illustrative embodiment, the electrodes of the electrode array 180 can be about 50 μm in width. In some embodiments, the electrodes of the electrode array 180 can be spaced by about 200 μm (i.e., 0.2 mm) to about 3,000 μm (i.e., 3 mm). In illustrative one embodiment, adjacent electrodes of the electrode array 180 can be spaced by about 400 μm.

The neural device 110 can further include a flexible substrate 212 supporting the electrode array 180 and/or other components of the neural device 110. In some embodiments, the flexible substrate 212 can be flexible enough to permit the electrode array 180 to be inserted through an osteotomy into the subdural space 204, then along the cortical surface.

The neural device 110 can include a range of electrical or electronic components. In the illustrated embodiment, the neural device 110 includes an electrode-amplifier stage 112, an analog front-end stage 114, an analog-to-digital converter (ADC) stage 116, a digital signal processing (DSP) stage 118, and a transceiver stage 120 that are communicatively coupled together. The electrode-amplifier stage 112 can include an electrode array, such as is described below, that is able to physically interface with the brain 102 of the subject in order to sense brain signals and/or apply electrical signals thereto. The analog front-end stage 114 can be configured, amplify signals that are sensed from or applied to the brain 102, perform conditioning of the sensed or applied analog signals, perform analog filtering, and so on. The front-end stage 114 can include, for example, one or more application-specific integrated circuits (ASICs) or other electronics. The ADC stage 116 can be configured to convert received analog signals to digital signals. The DSP stage 118 can be configured to perform various DSP techniques, including multiplexing of digital signals received via the electrode-amplifier stage 112 and/or from the external device 130. For example, the DSP stage 118 can be configured to convert instructions from the external device 130 to a corresponding digital signal. The transceiver stage 120 can be configured to transfer data from the neural device 110 to the external device 130 located outside of the body of the subject 102.

In some embodiments, the neural device 110 can further include a controller 119 that is configured to perform various functions, including compressing electrophysiologic data generated by the electrode array 180. In various embodiments, the controller 119 can include hardware, software, firmware, or various combinations thereof that are operable to execute the functions described below. In one embodiment, the controller 119 can include a processor (e.g., a microprocessor) executing instructions stored in a memory. In another embodiment, the controller 119 can include a field-programmable gate array (FPGA) or application-specific integrated circuit (ASIC).

In various embodiments, the stages of the neural device 110 can provide unidirectional or bidirectional communications (as indicated in FIG. 1) by and between the neural device 110 and the external device 130. In various embodiments, one or more of the stages can operate in a serial or parallel manner with other stages of the system 100. It can further be noted that the depicted architecture for the system 100 is simply intended for illustrative purposes and that the system 100 can be arranged differently (i.e., components or stages can be connected in different manners) or include additional components or stages.

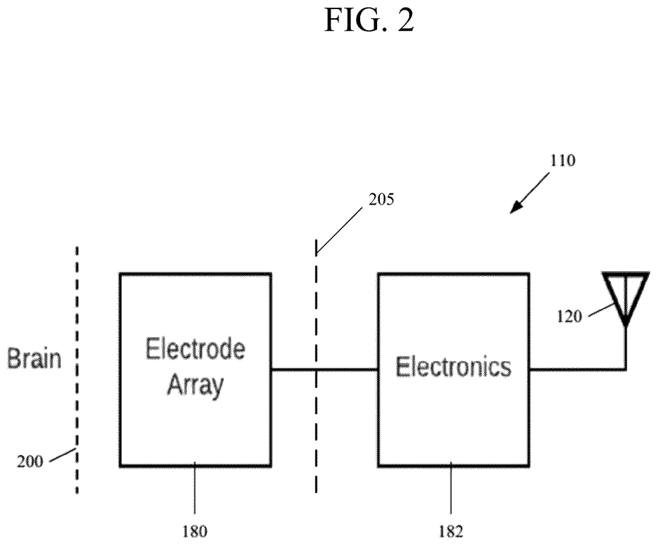

In some embodiments, the neural device 110 described above can include a brain implant, such as is shown in FIG. 2. The neural device 110 may be a biomedical device configured to study, investigate, diagnose, treat, and/or augment brain activity. In some embodiments, the neural device 110 may be positioned between the brain 200 and the scalp 202. The neural device 110 can include an electrode array 180 (which may be a component of or coupled to the electrode-amplifier stage 112 described above) that is configured to record and/or stimulate an area of the brain 200. The electrode array 180 can be connected to an electronics hub 182 (which can include one or more of the electrode-amplifier stage 112, analog front-end stage 114, ADC stage 116, and DSP stage 118) that is configured to transmit via wireless or wired transceiver 120 to the external device 130 (in some cases, referred to as a “receiver”).

The electrode array 180 can include non-penetrating cortical surface microelectrodes (i.e., the electrode array 180 does not penetrate the brain 200). Accordingly, the neural device 110 can provide a high spatialresolution, with minimal invasiveness and improved signal quality. The minimal invasiveness of the electrode array 180 is beneficial because it allows the neural device 110 to be used with larger population of patients than conventional brain implants, thereby expanding the application of the neural device 110 and allowing more individuals to benefit from brain-computer interface technologies. Furthermore, the surgical procedures for implanting the neural devices 110 are minimally invasive, reversible, and avoid damaging neural tissue. In some embodiments, the electrode array 180 can be a high-density microelectrode array that provides smaller features and improved spatial resolution relative to conventional neural implants.

In some embodiments, the neural device 110 includes an electrode array configured to stimulate or record from neural tissue adjacent to the electrode array, and an integrated circuit in electrical communication with the electrode array, the integrated circuit having an analog-to-digital converter (ADC) producing digitized electrical signal output. In some embodiments, the ADC or other electronic components of the neural device 110 can include an encryption module, such as is described below. The neural device 110 can also include a wireless transmitter (e.g., the transceiver 120) communicatively coupled to the integrated circuit or the encryption module and an external device 130. The neural device 110 can also include, for example, control logic for operating the integrated circuit or electrode array 180, memory for storing recordings from the electrode array, and a power management unit for providing power to the integrated circuit or electrode array 180.

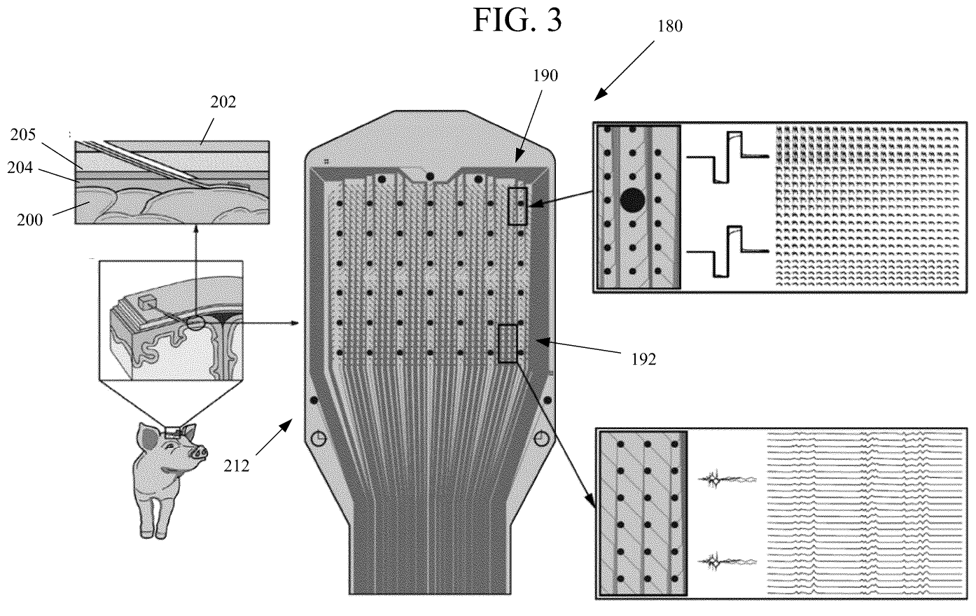

Referring now to FIG. 3, there is shown a diagram of an illustrative embodiment of a neural device 110. In this embodiment, the neural device 110 comprises an electrode array 180 comprising nonpenetrating microelectrodes. As generally described above, the neural device 110 is configured for minimally invasive subdural implantation using a cranial micro-slit technique, i.e., is inserted into the subdural space 204 between the dura and the surface of the subject's brain 200. In some embodiments, the neural device 110 is inserted into the subdural space 204 between the dura and the surface of the brain 200. Further, the microelectrodes of the electrode array 180 can be arranged in a variety of different configurations and can vary in size. In this particular example, the electrode array 180 includes a first group 190 of electrodes (e.g., 200 μm microelectrodes) and a second group 192 of electrodes (e.g., 20 μm microelectrodes). Further, example stimulation waveforms in connection with the first group 190 of electrodes and the resulting post-stimulus activity recorded over the entire array is depicted for illustrative purposes. Still further, example traces from recorded neural activity recorded by the second group 192 of electrodes are likewise illustrated. In this example, the electrode array 180 provides multichannel data that can be used in a variety of electrophysiologic paradigms to perform neural recording of both spontaneous and stimulus-evoked neural activity as well as decoding and focal stimulation of neural activity across a variety of functional brain regions.

Additional information regarding brain-computer interfaces described herein can be found in Ho et al., The Layer 7 Cortical Interface: A Scalable and Minimally Invasive Brain-Computer Interface Platform, bioRxiv 2022.01.02.474656; doi: https://doi.org/10.1101/2022.01.02.474656, which is hereby incorporated by reference herein in its entirety.

### Minimally Invasive Insertion Technique for Neural Devices

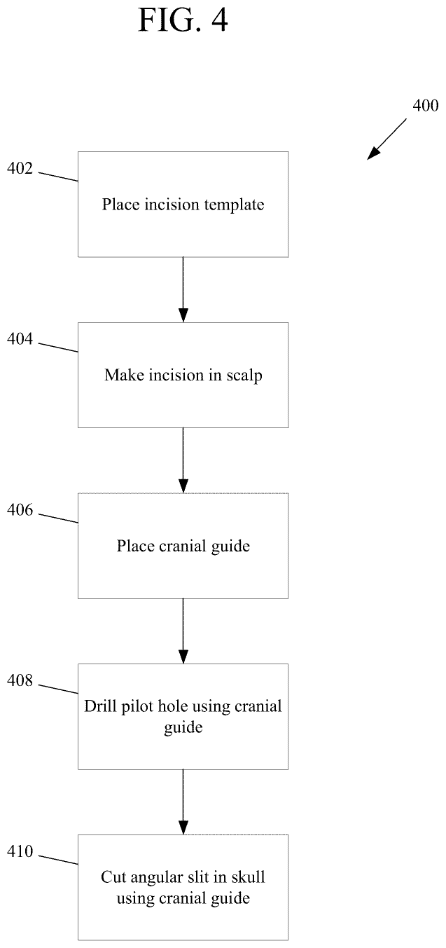

As generally described above, the neural device 110 is designed to be surgically inserted into a patient in a noninvasive manner. In one embodiment, the neural device 110 can be inserted using a minimally invasive “cranial micro-slit” technique. A flowchart illustrating the cranial micro-slit technique process 400 is shown in FIG. 4. In various embodiments, the process 400 can be formed by, for example, one or more members of a surgical team (e.g., a surgeon) a robotic surgical system.

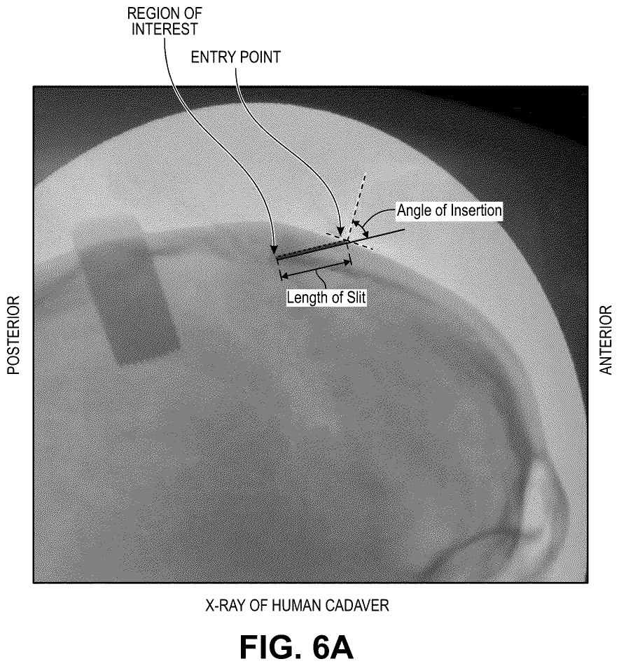

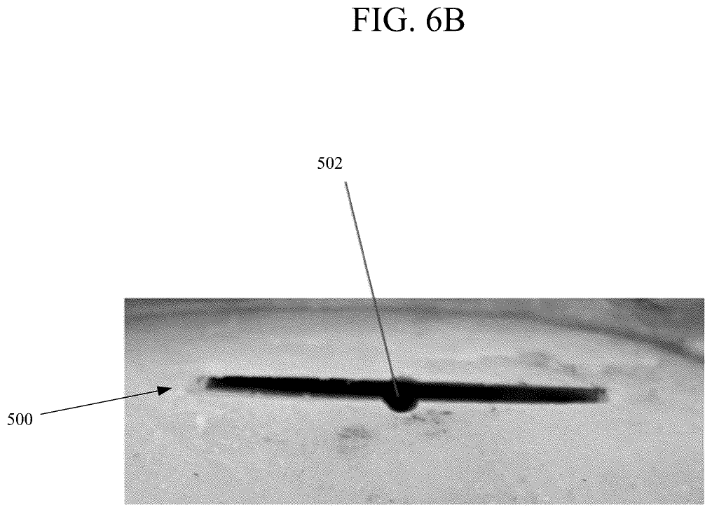

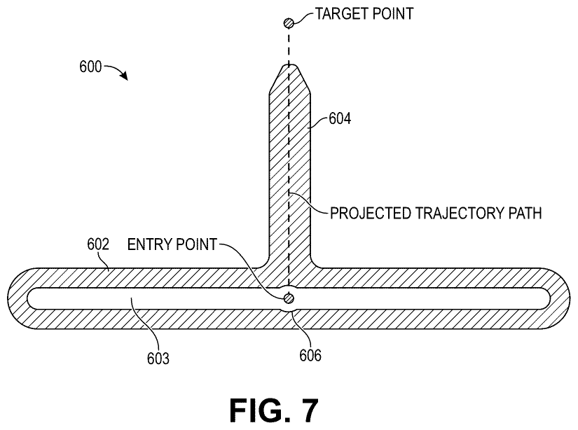

Initially, the surgical team can place 402 an incision template against the patient's scalp. The incision template can be configured to guide the entry point and trajectory for the insertion of the neural device 110. One embodiment of an incision template 600 is shown in FIG. 7 and described in greater detail below. Using the incision template as a guide, the surgical team can make 404 an incision in the scalp. The incision can be made 404 via, for example, a freehand scalpel with suture retraction. The incision template can be configured to mark the placement of the skin incision and cranial guide for the planned trajectory between the target location for the neural device 110 against the cortical surface and the entry point of the patient's skull, as shown in FIG. 6A.

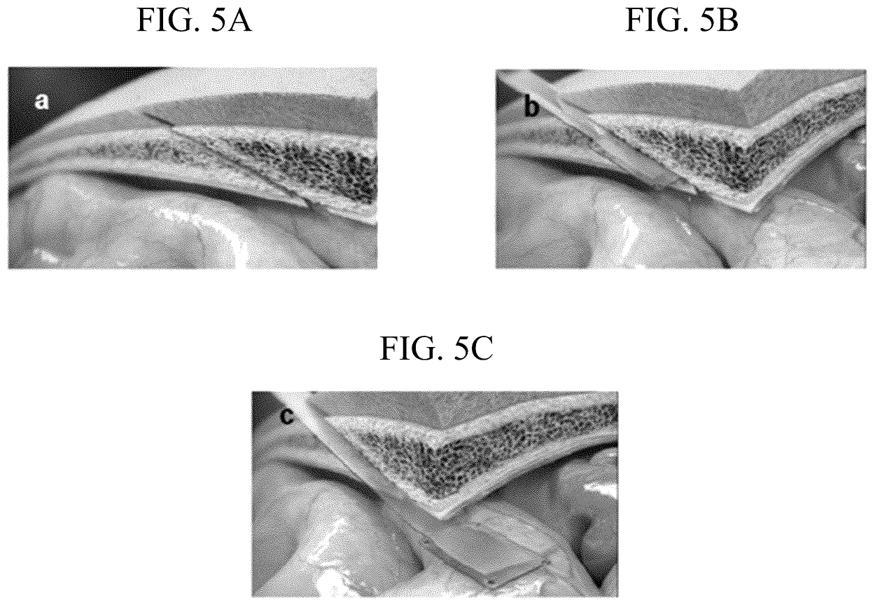

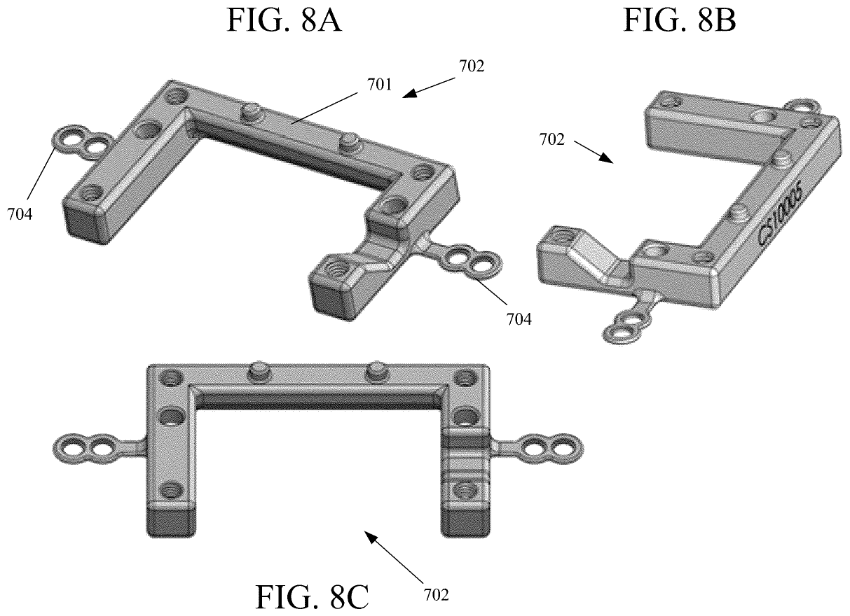

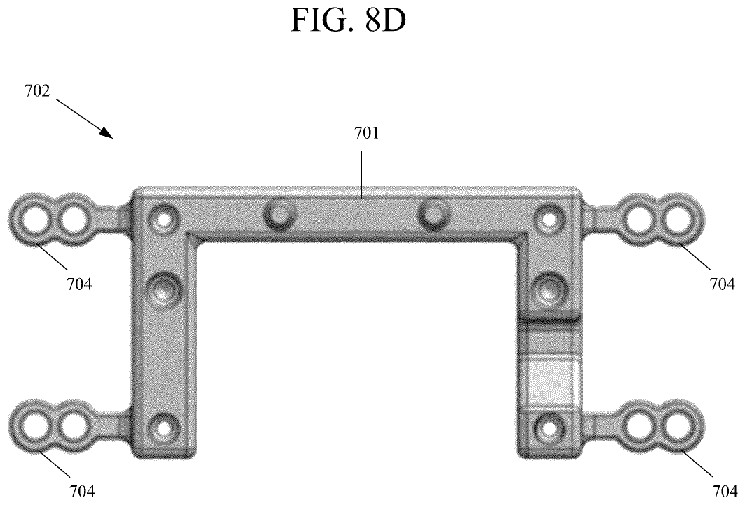

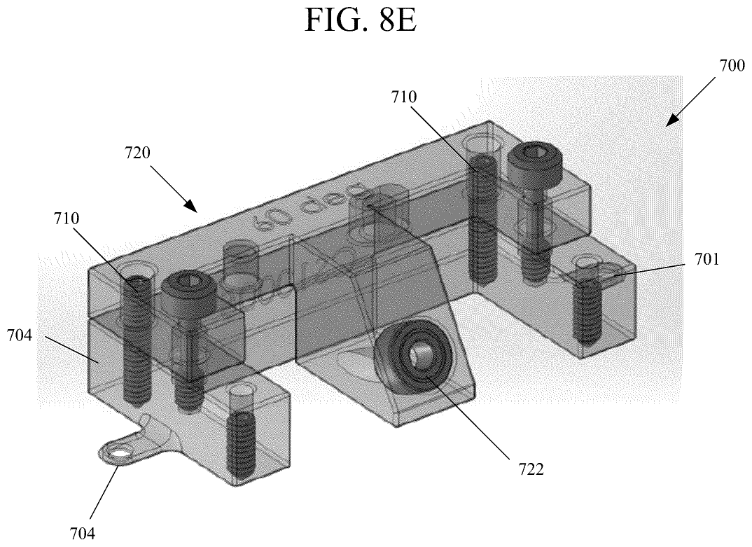

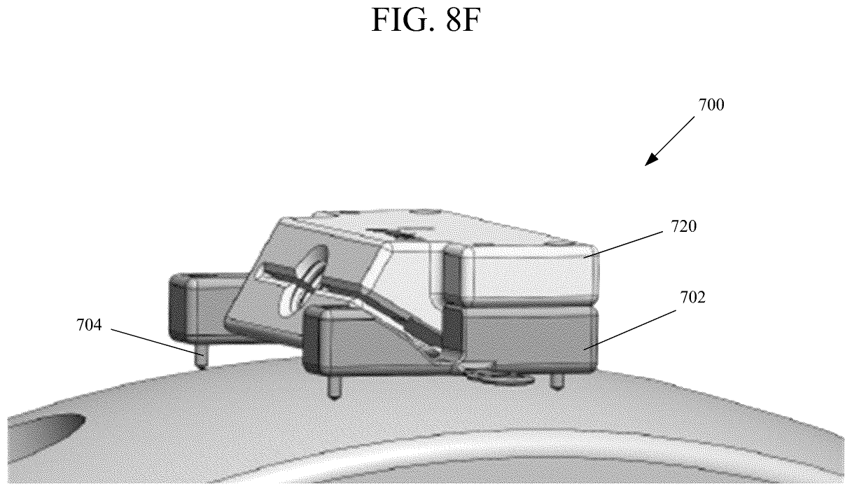

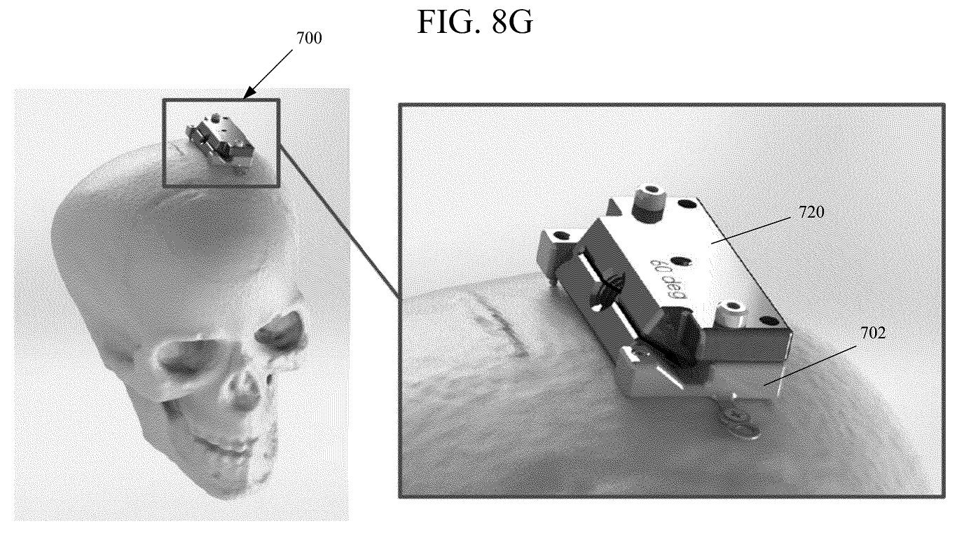

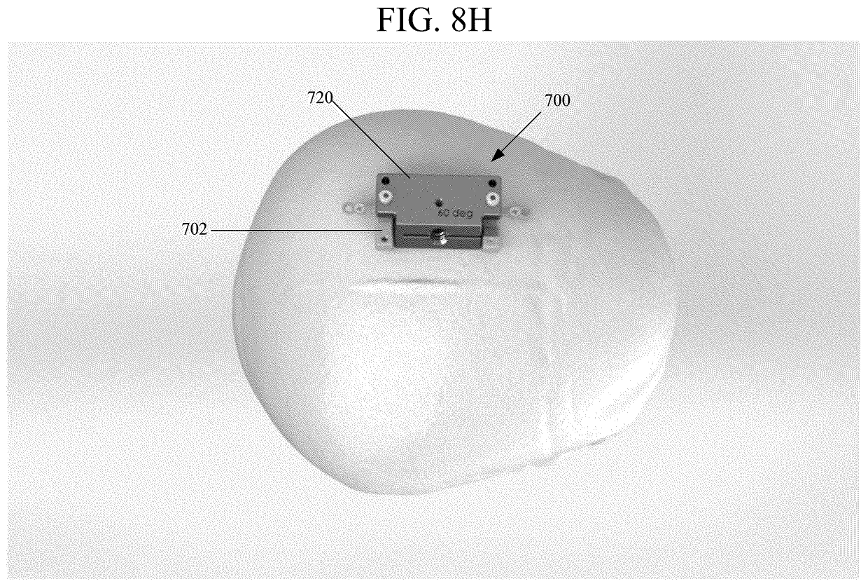

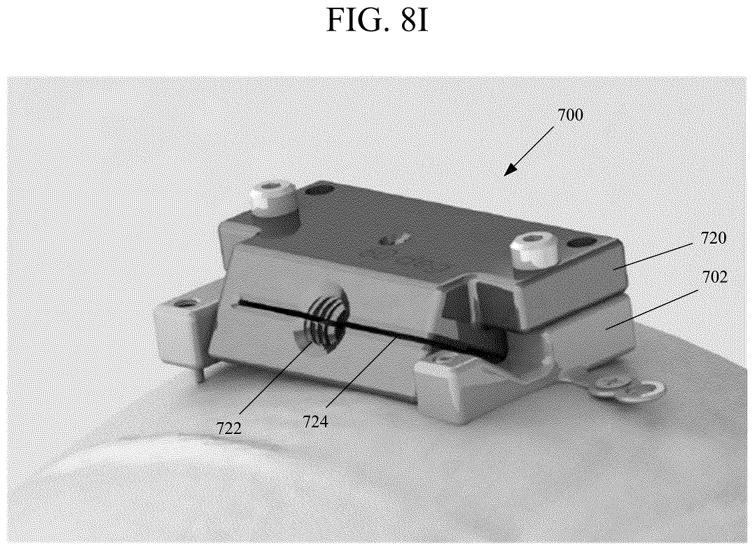

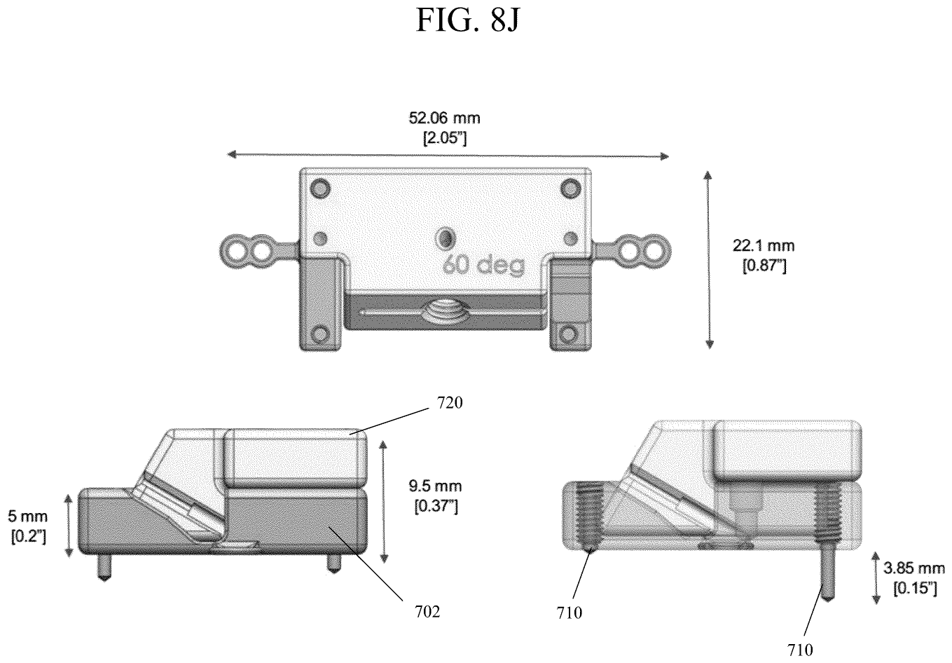

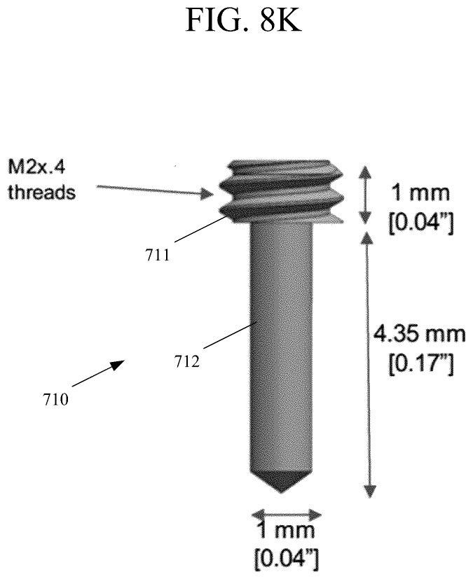

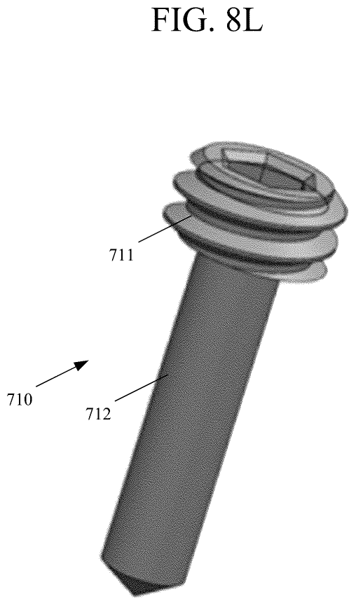

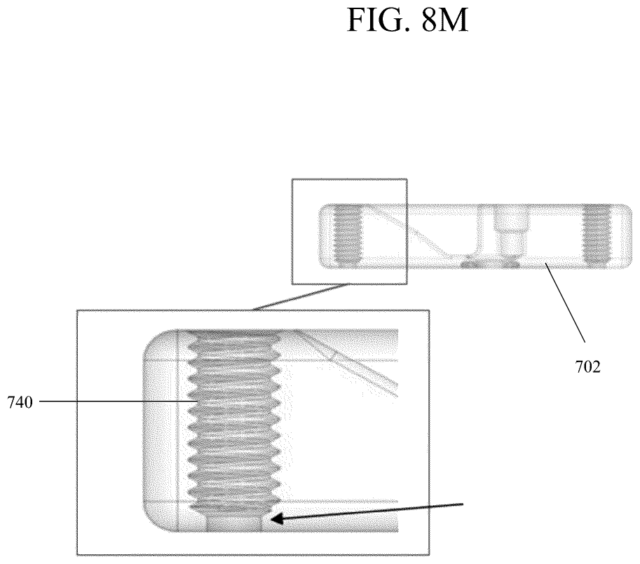

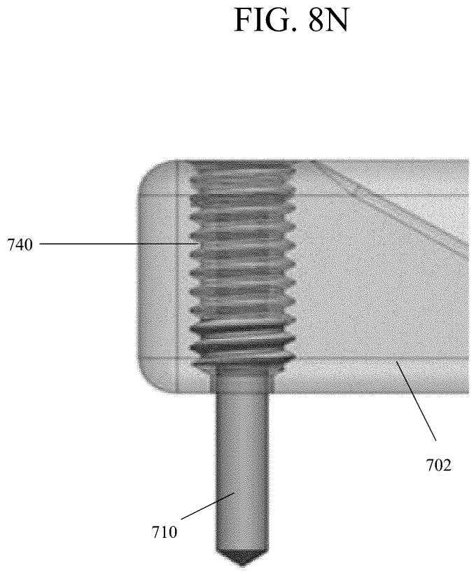

After the incision is made 404, the surgical team can place 406 the cranial guide, drill 408 a pilot hole 502 (FIG. 6B) guided by the cranial guide, and cut 410 an angular slit 500 (FIG. 6B) in the patient's skull guided by the cranial guide, as well as making a dural incision to access the subdural space. The angular slit 500 can allow the neural device 110 to be inserted at an approach angle that it is tangential to the cortical surface in order to facilitate subdural insertion of the neural device 110, as shown in FIGS. 5A-5C. One embodiment of a cranial guide system 700 is shown in FIGS. 8A-16 and described in greater detail below. The angular slit 500 can be made via, for example, a sagittal saw. As illustrated in FIG. 6A, the length and angle of the angular slit 500 can be pre-planned based on the location of the region of interest on the cortical surface and the desired angle of insertion for the neural device 110. In various embodiments, the length of the angular slit 500 can be from about 500 μm to about 900 μm. In various embodiments, the angle of the angular slit 500 can be from about 45° to about 65°.

As shown in FIGS. 5A-5C, the neural device 110 can be deployed into the subdural space to the targeted cortical region of the brain. In particular, FIG. 5A depicts a complete skull and dural incision to the target region on the brain created by, for example, the process 400 described above. FIG. 5B depicts the deployment of the neural device 110 through the osteotomy created according to the process 400 on approach to the dural incision. The neural device 110 is deployed using a semi-flexible stylet that is sufficiently stiff or rigid to allow the neural device 110 to be pushed through the osteotomy and dural incision, but also sufficiently flexible to allow the stylet to bend and conform to the cortical surface to deploy the neural device 110 thereagainst. FIG. 5C depicts complete deployment of the neural device 110 at or on the target region of the cortical surface. The neural device 110 comprises a pocket, described above, that is shaped and otherwise configured to receive the semi-flexible stylet. Once the neural device 110 is positioned at the target region of the cortical surface, the stylet can be retracted back through the osteotomy, removing the stylet from the pocket of the neural device 110, while maintaining the neural device 110 at the target region.

In some embodiments, trajectory planning and insertion can be performed using fluoroscopy or computed tomographic image guidance. Further, in some embodiments, electrode insertion can be monitored using neuroendoscopy.

### Surgical System for Minimally Invasive Insertion of Neural Devices

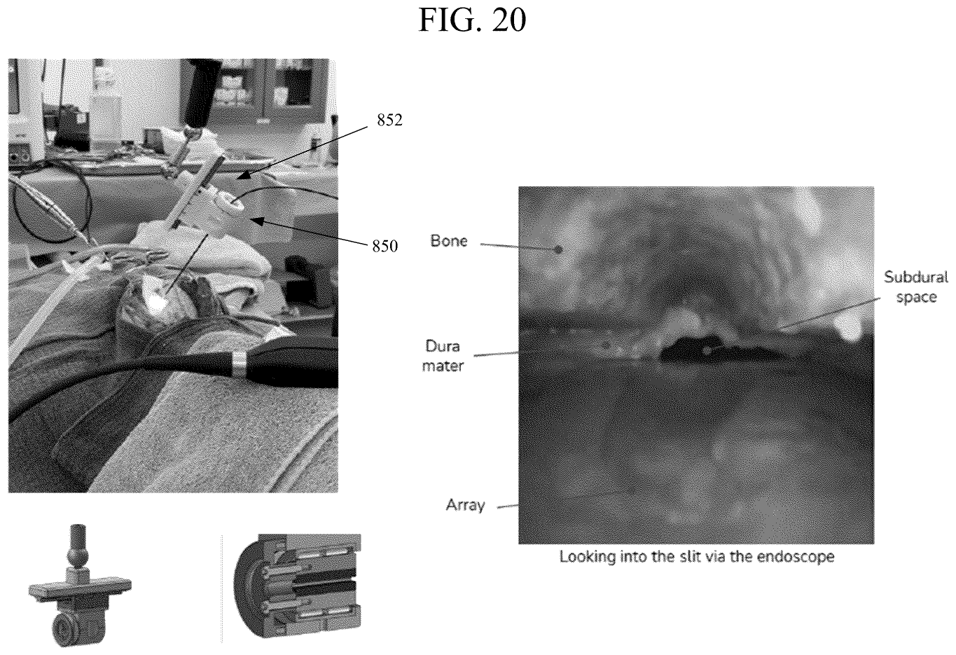

The processes and techniques described above for implanting the neural devices 110 can make use of a number of different surgical instruments and devices. Various embodiments of such surgical instruments and devices that make up the surgical system are described herein. Various embodiments of the surgical instruments and/or devices are described below and illustrated in FIGS. 7-25C. It should be understood that the medical instruments and/or devices described below can be used in connection with the processes and techniques described above. However, the medical instruments and/or devices described below can additionally be used in connection with different surgical techniques and/or processes or in the performance of different steps of the surgical techniques and/or processes described above. The medical instruments and/or devices can have a variety of different uses and should not be understood to be limited solely to the described surgical techniques and/or processes, or to any individual steps thereof.

### Incision Template

As described above, an incision template can be used to plan and guide the initial incision made into the patient's scalp, which in turn corresponds to where the pilot hole 502 and angular slit 500 will be formed in the patient's skull. One embodiment of an incision template 600 is shown in FIG. 7. The incision template 600 can have a generally T-shaped structure including a first portion 602 and a second portion 604 arranged orthogonally with respect to the first portion 602. In one embodiment, the second portion 604 can extend from a midpoint of the first portion 602. The first portion 602 can define a slit 603 that is sized, shaped, and otherwise configured to receive a scalpel or other such cutting instrument therethrough. The length of the slit 603 can correspond to the length of the angular slit 500. In some embodiments, the length of the slit 603 can be from about 500 μm to about 900 μm. The slit 603 can further include a substantially circular opening 606 that is aligned with the longitudinal axis of the second portion 604. The circular opening 606 can be sized, shaped, and otherwise configured to receive a drill bit therethrough. The circular opening 606 can define the entry point for the pilot hole 502, which can be located at a midpoint of the angular slit 500.

Accordingly, the incision template 600 can be used by aligning the distal end of the second portion 604 with the target point on the cortical surface. As noted above, fluoroscopy, computed tomography, and other imaging modalities can be used for imaging the cortical surface to aid in aligning the incision template 600 with the target point. Once positioned properly, the incision template 600 can be used to mark the patient's scalp with the locations for the scalp incision (and, accordingly, the angular slit 500 through the patient's skull) and the drilled pilot hole 502. Thereafter, the scalp incision can be made via, for example, a scalpel. Suture retraction, anchors, and/or other securement techniques can be used to maintain the scalp incision opening for the surgical team.

### Cranial Guide System

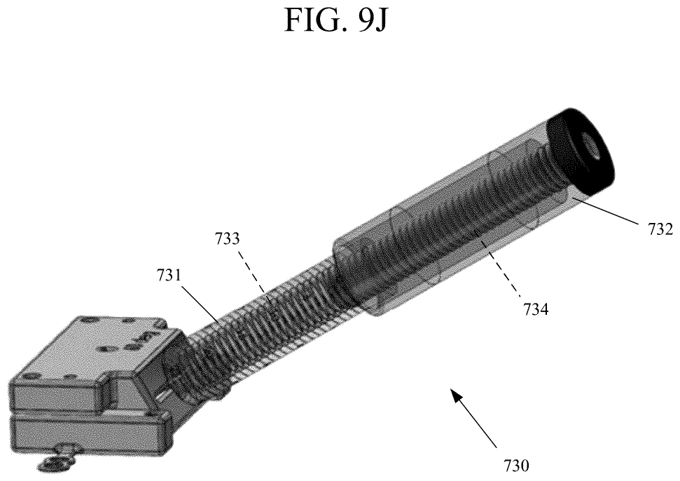

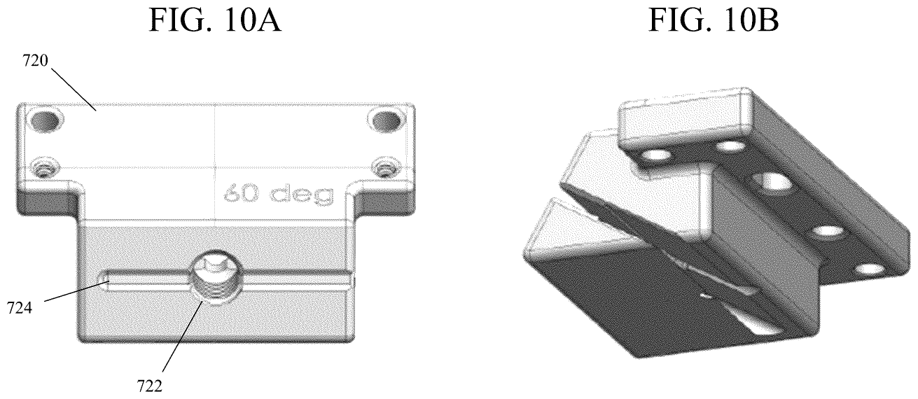

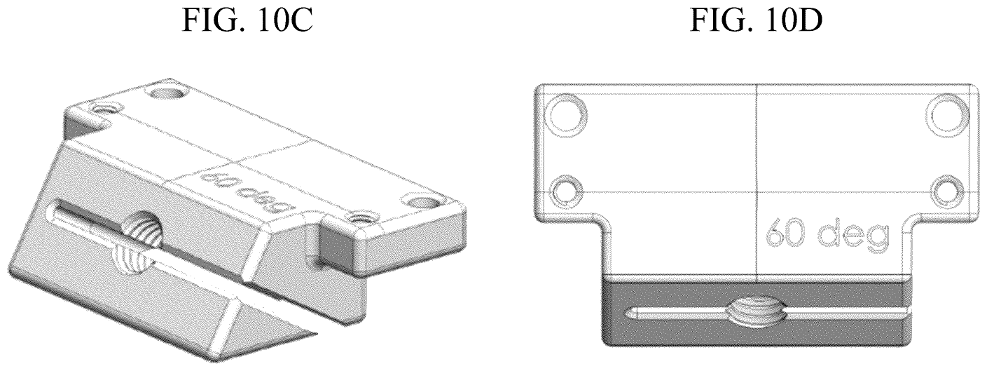

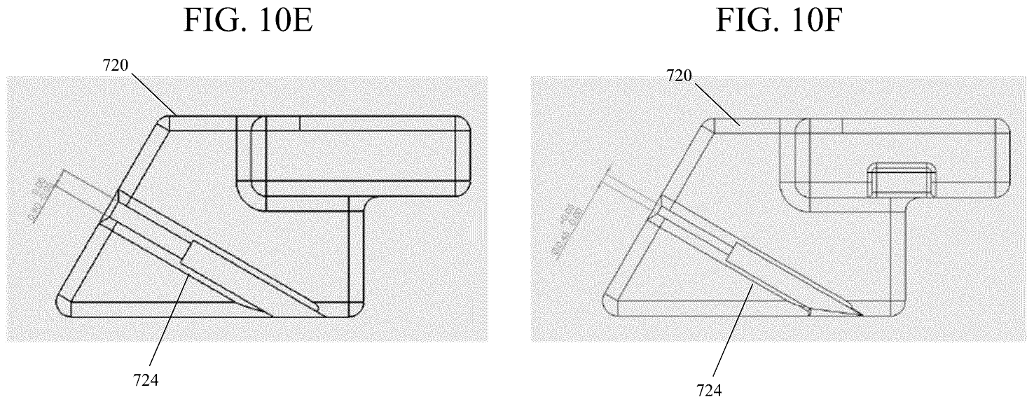

As described above, an incision template can be used to guide the creation of the pilot hole 502 and the angular slit 500 in the patient's skull. Various embodiments of a cranial guide system 700 is shown in FIGS. 7-16. In operation, the cranial guide system 700 can alternatively serve as a guide for drilling the pilot hole 502 and a jig for guiding the cutting of the angular slit 500 (e.g., via a sagittal saw). The cranial guide can include a variety of different components, including a base or mount 702 (FIGS. 8A-8D) and a guide portion 720 (FIGS. 10A-10F) that is removably securable to the mount 702. In one illustrative embodiment shown in FIG. 8J, the cranial guide can be about 52 mm in length, about 22 mm in width, and about 9.6 mm in height when the guide portion 720 is assembled to the mount 702. In this illustrative embodiment, the mount 702 can be about 5 mm in height without the guide portion affixed thereto. As discussed in greater detail below, the cranial guide can further include adjustable leveling pins 710 for controlling the position and/or orientation of the cranial guide with respect to the subject. The leveling pins 710 can be adjustable from an undeployed position whereby the leveling pin 710 is flush with the bottom surface of the mount 702 (as shown by the left leveling pin 710 in the bottom right view in FIG. 8J) to a deployed position, or any position therebetween. In one illustrative embodiment, the length of a leveling pin 710 can be about 3.85 mm in the deployed position.

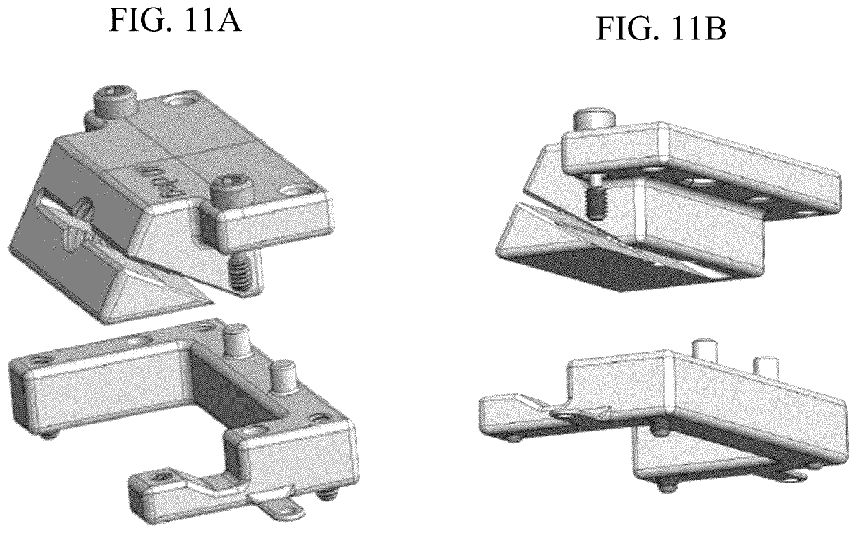

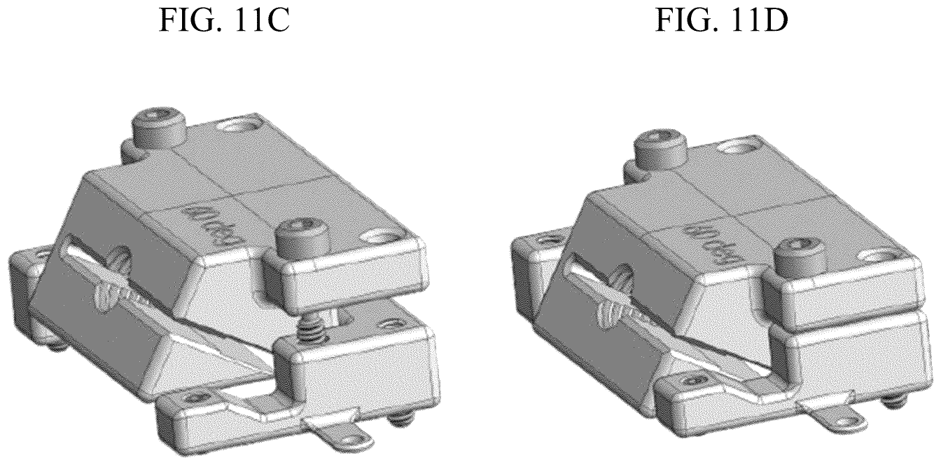

Referring now to FIGS. 8A-8C, there is shown an embodiment of the cranial guide mount 702. The mount 702 can include frame 701 that is configured to receive the guide portion 720 (described below) and feet 704 that are configured to receive fasteners (e.g., screws) therethrough for securing the mount 702 to the patient's skull. As shown in FIGS. 8E-8I and FIGS. 11A-11D, the guide portion 720 can in turn be secured to the mount 702 via fasteners (e.g., screws). The mount 702 can include a variety of different numbers of feet 704 for securing the mount 702 to the subject's skull, such as is shown in FIGS. 8G and 8H. In the embodiment shown in FIGS. 8A-8C, the mount 702 can include two feet 704. In an alternative embodiment shown in FIG. 8D, the mount 702 can include four feet 704.

In one embodiment, the mount 702 can further include leveling pins 710 that are configured to control the position and/or orientation of the mount 702 and the guide portion 720 relative to the subject. Accordingly, the leveling pins 710 allow the surgical team to adjust the positioning and orientation of the drill aperture 722 (FIGS. 10A-10F) and saw slot 724 (FIGS. 10A-10F) in order to ensure that the pilot hole 502 and angled slit are formed with the correct positioning and orientation for the target location and approach angle. In one embodiment, the drill aperture 722 can be sized to accommodate drill sizes from about 0.5 mm to about 3 mm. In one embodiment, the saw slot 724 can be sized to accommodate one or more of the neural interface electrode arrays described above (e.g., 1,024 channel electrode arrays) therethrough.

In one embodiment shown in FIGS. 8K-8N, the leveling pins 710 be screw-based pins having threading that allows the leveling pins 710 to be extended or retracted from corresponding threaded channels 740 disposed on the mount 702. In this embodiment, the leveling pins 710 can include a threaded portion 711 and an elongated body portion 712. The threaded portion 711 can be configured to engage with a corresponding threaded channel 740 (FIGS. 8M and 8N) extending through the mount 702. Due to the threaded engagement between the leveling pin threaded portion 711 and the mount threaded channel 740, the leveling pin 710 can be adjustably or selectively extended or retracted from the threaded channel 740. When the cranial guide is affixed to the subject's skull, the leveling pins 710 can be extended or otherwise adjusted to bear against the subject's anatomy, thereby stabilizing the cranial guide in place and preventing the cranial guide from tilting or rotating during the surgical procedure. In one illustrative embodiment shown in FIG. 8K, the threaded portion 711 can be about 1 mm in length, the body portion 712 can be about 4.35 mm in length, and the body portion 712 can be about 1 mm in width. In other embodiments, the leveling pins 710 can be adjustably extended and/or retracted using a variety of other mechanisms.

Referring back to FIG. 8E, in one embodiment, the guide portion 720 can include a drill aperture 722 configured to receive a drill therethrough for forming the pilot hole 502. In another embodiment shown in FIGS. 8F-8I and 10A-16, the guide portion 720 can include both the drill aperture 722 and the saw slot 724 for guiding the cutting of the angled slit in the patient's skull. In one embodiment, the angle of the drill aperture 722 can correspond to the desired approach angle for the delivering of the neural device. Likewise, in one embodiment, the angle of the saw slot 724 can correspond to the desired approach angle for the delivering of the neural device. Accordingly, in various embodiments, the angle of the drill aperture 722 and/or saw slot 724 can be from about 45° to about 65°; accordingly, the guide portion 720 can be configured to accommodate surgical approach angles ranging from about 45° to about 65°. For example, FIG. 8O depicts various illustrative embodiments of the guide portion 710 wherein the drill aperture 722 and the saw slot 724 are oriented at 45°, 50°, 55°, and 65°. In one embodiment, the drill aperture 722 and the saw slot 724 can be coextensive such that the pilot hole 502 is correspondingly formed so that it is coextensive with the angular slit 500. In some embodiments, the cranial guide system 700 can be manufactured having a variety of different approach angles. In other embodiments, the cranial guide system 700 can be configured such that it can be adjustable between a variety of different approach angles.

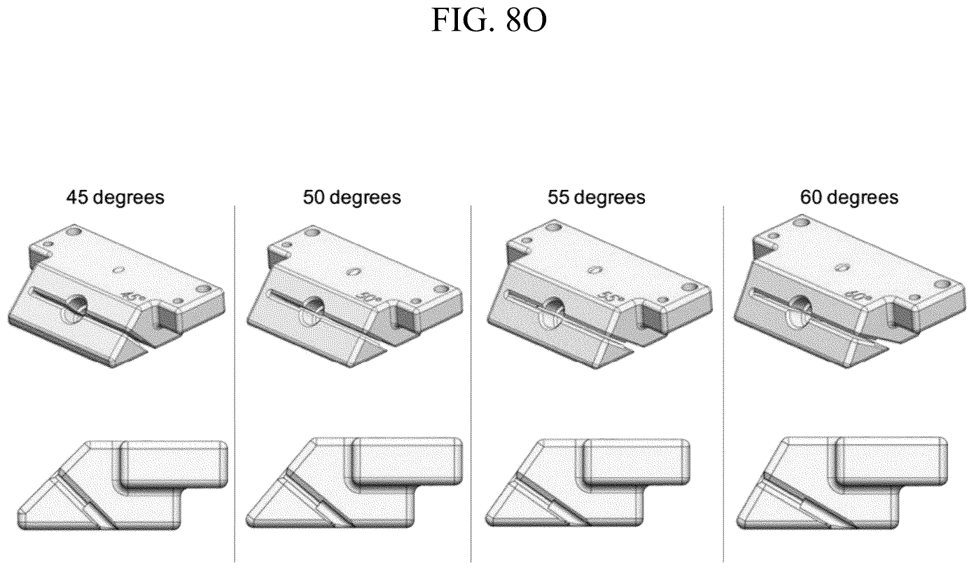

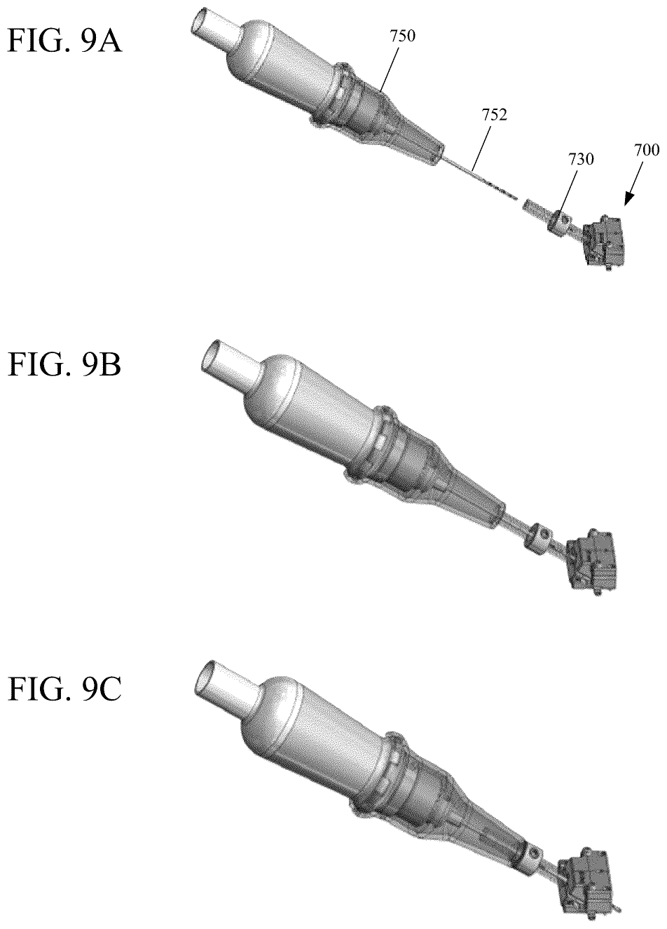

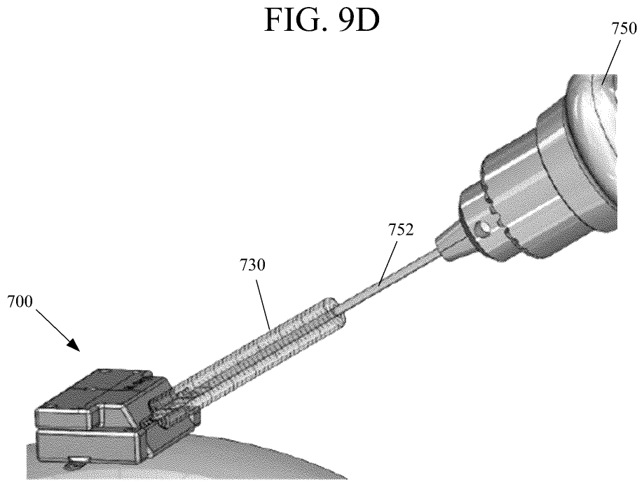

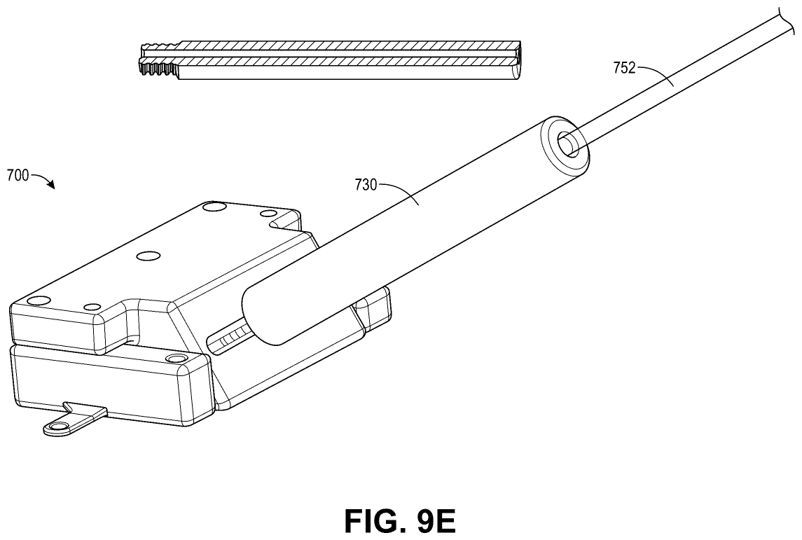

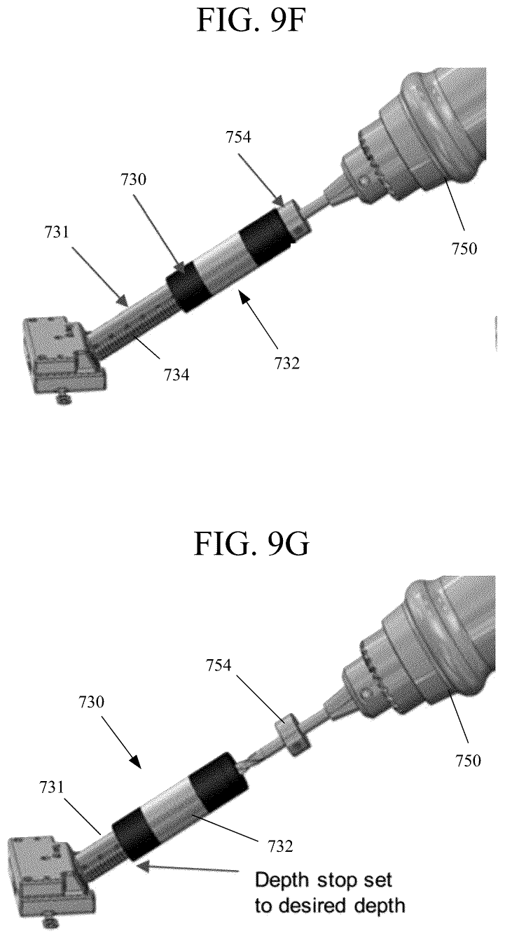

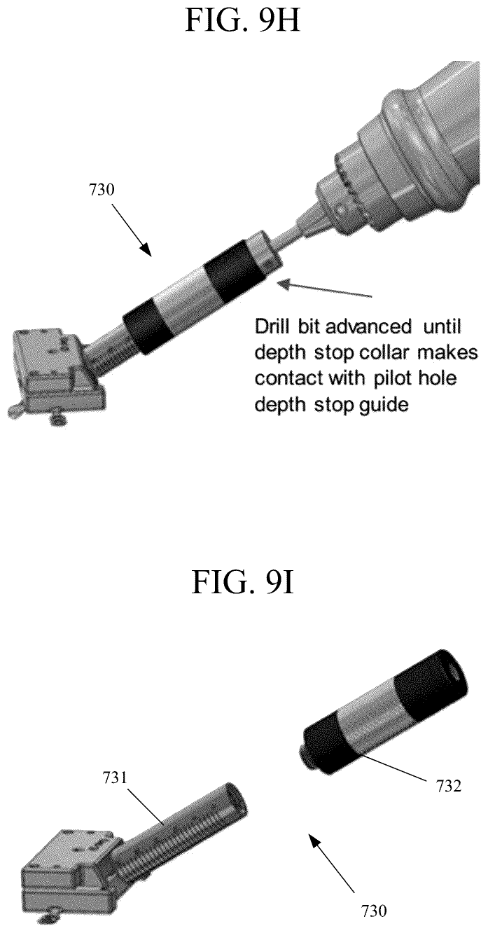

The cranial guide system 700 can further include or be used with a drill stop 730 that is configured to stop the drill bit at a predetermined depth, thereby ensuring that the drill bit goes through the skull but does not contact the cortical surface. In one embodiment, the drill stop 730 can include a collar that is fitted to, or otherwise associated with, the drill aperture 722. The drill stop collar can have a predetermined length that physically stops the drill, thereby preventing the drill bit from being advanced further through the drill guide 700. Referring now to FIGS. 9A-9C, there are shown views of the cranial guide system 700 being used to drill the pilot hole 502, in association with the techniques as described above. In particular, FIG. 9A illustrates a drill 750 having an unloaded drill bit 752 with drill stop 730 and the cranial guide system 700. In FIG. 9B, the drill bit 752 has been loaded into cranial guide system 700 to begin drilling the pilot hole 502. In FIG. 9C, the drill 750 has contacted the stop 730. Accordingly, the drill bit 752 is at its final position as the drill 750 makes contact with the drill stop 730 and prevents further drilling.

In one embodiment, the cranial guide system 700 can include an adjustable depth stop 730, as shown in FIGS. 9F-J. In this embodiment, the drill stop 730 can include a first or interior portion 731 that is configured to engage with a second or exterior portion 732. The first and second portions 731, 732 include an interior channel having a tight toleranc interior diameter that corresponds to the drill bit. The tight tolerance internal diameter can be configured to contact the shaft of the drill bit to keep the bit concentric to the trajectory of the angle guide. Further, the first portion 731 can include a threaded conduit 733 (FIG. 9J) that is configured to engage with a corresponding threaded portion 734 of the second portion 732. Due to the threaded engagement between the components, rotating the second portion 732 relative to the first portion 731 (i.e., screwing or unscrewing the second portion 732 from the first portion 731) allows one to adjust the relative position of the second portion 732 with respect to the first portion 731. Accordingly, the drill stop 730 can be set to different positions (as shown in FIGS. 9F and 9G). The second portion 732 can serve as a physical stop that contacts the collar 754 of the drill 750, thereby preventing the drill 750 from being inserted further into the cranial guide system 700. Accordingly, by setting the drill stop 730 to different positions, one set different depth stops for the drill 750. In one embodiment, the first portion 731 can further include depth markings 734 indicating the depth to which the drill stop 730 is set. Accordingly, the drill stop 730 allows for fine (e.g., millimeter-scale) adjustment to be made to the drill depth for the drill, without requiring any tools.

In some embodiments shown in FIGS. 9D and 9E, various components of the drill 750 and/or drill system can include various coatings at contact points between the components to reduce friction and the creation of metallic debris. Reducing friction between the components can be desirable in order to extend the durability of the components. Further, reducing or preventing the creation of debris can be desirable to prevent the debris from entering the surgical field (e.g., into the patient). The coatings can include various materials that have been approved for use in surgical applications. For example, the coatings can include polytetrafluoroethylene, Xylan® 8110, Eclipse®, plasma polymerized coating, diamond-like carbon (DLC) coating, lubricious silicone coating, and any combination thereof. In some embodiments, the thickness of the coating can be from about 2 μm to about 80 μm. For example, the drill bit 752 can include one or more coatings. As another example shown in FIG. 9E, the interior surface of the drill stop 730 can include one or more coatings.

A variety of different drills 750 and/or drill bit 752 can be used with the cranial guide system 700 described herein. For example, the drill 750 can include Midas Rex Jacobs Chuck handpiece from Medtronic®. As another example, the drill bit 752 can include a 1/16^{th} inch, extended length, drill bit.

Referring now to FIGS. 10A-10F, there are shown various views of the guide portion 720 of the cranial guide system 700. As generally noted above, the guide portion 720 can include a drill aperture 722 that is coextensive with a saw slot 724. In one embodiment, the drill aperture 722 can be located at a midpoint of the saw slot 724. The saw slot 724 is configured to receive one or more saw blades (e.g., a sagittal saw blade) therethrough. The width of the saw slot 724 can correspond to the number of saw blades that the saw slot 724 is configured to receive. In some embodiments, it can be beneficial to use different numbers of saw blades in order to make differently sized osteotomies. For example, FIG. 10E illustrates an embodiment of the guide portion 720 wherein the saw slot 724 has a thickness that is sufficient to receive two saw blades. Accordingly, this embodiment allows two stacked saw blades to be inserted through the saw slot 724 for cutting a double width osteotomy. As another example, FIG. 10F illustrates an embodiment of the guide portion 720 wherein the saw slot 724 has a width that is sufficient to receive a single saw blade. Accordingly, this embodiment allows a single saw blade to be inserted through the saw slot 724 for cutting a smaller osteotomy. In one embodiment, the thickness of the saw slot 724 can be from about 0.4 mm to about 0.8 mm. In another embodiment, the thickness of the saw slot 724 can be from about 0.8 mm to about 1 mm.

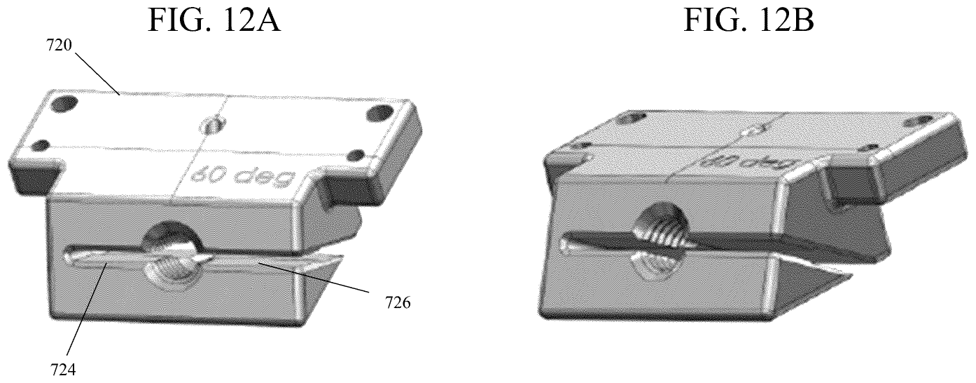

In some embodiments shown in FIGS. 12A and 12B, various components of the guide portion 720 can include various coatings at contact points between the drill 750 and/or components of the cranial guide system 700. As noted above, coatings can be beneficial in order to reduce friction and the creation of metallic debris. The coatings can include various materials that have been approved for use in surgical applications. For example, the coatings can include polytetrafluoroethylene, Xylan® 8110, Eclipse®, plasma polymerized coating, diamond-like carbon (DLC) coating, lubricious silicone coating, and any combination thereof. In the embodiment shown in FIGS. 12A and 12B, the interior surface 726 of the saw slot 724 can include one or more coatings.

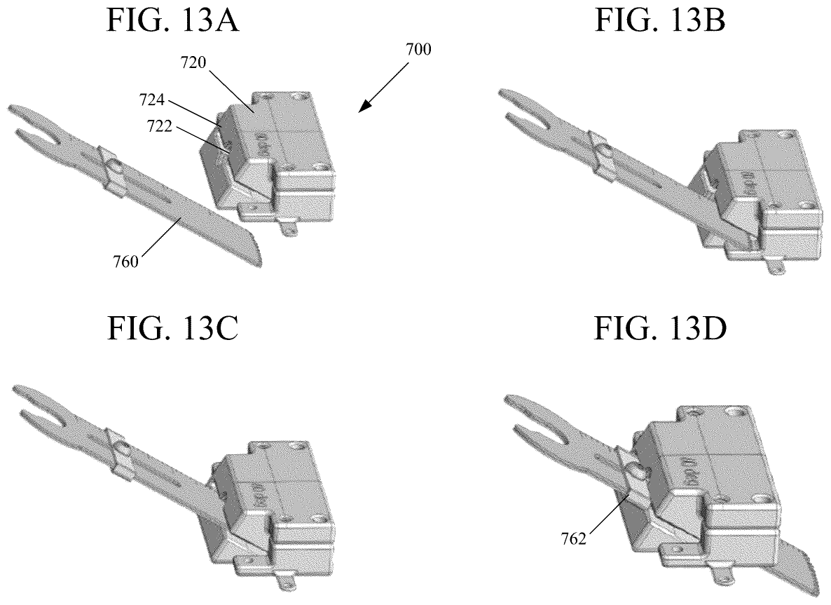

Referring now to FIGS. 13A-13D, there are shown views of a saw blade 760 being used in connection with the cranial guide system 700. In particular, FIG. 13A illustrates an oscillating saw blade 760 unloaded from the cranial guide system 700. FIG. 13B illustrates the oscillating saw blade 760 being initially inserted through the angled saw slot 724 of the cranial guide system 700. FIG. 13C illustrates the position of the oscillating saw blade 760 fully loaded into the cranial guide system 700 during creation of the angular slit 500. In some embodiments, the saw blade 760 can include a stop 762 that is configured to control the maximum depth to which the saw blade 760 can be inserted in order to control the depth of the angular slit 500 and, accordingly, prevent the saw blade 760 from contacting the cortical surface. FIG. 13D illustrates the final position of the oscillating saw blade 760 where the blade stop 762 is contacting the guide portion 720 to prevent further drilling.

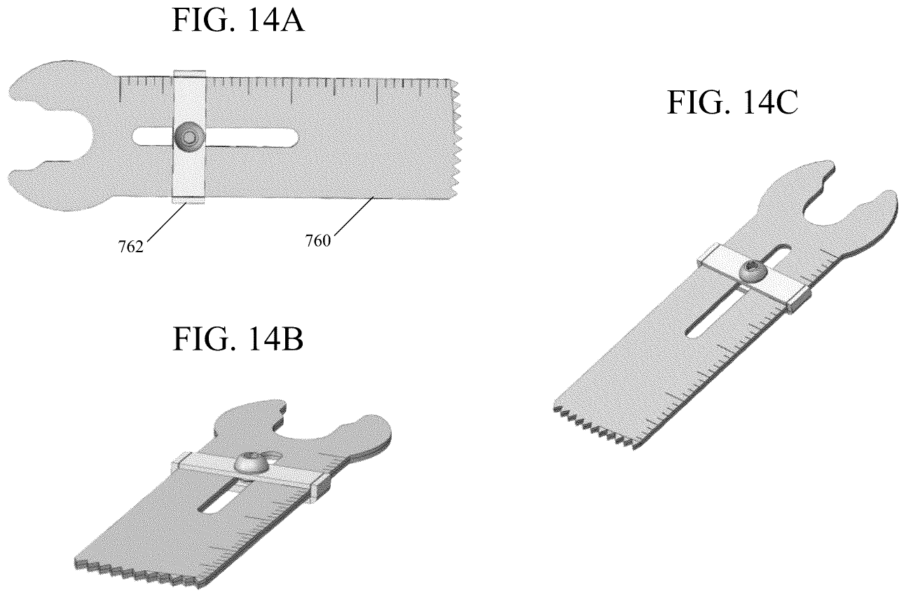

In one embodiment shown in FIGS. 14-14C, the blade stop 762 can be adjustable to allow the surgical team to control the depth of the angular slit 500. For example, the position of the blade stop 762 can be slidably adjustable along the length of the saw blade 760. Accordingly, the blade stop 762 can be positioned to set the saw blade 760 to the desired depth and then locked in place. In one embodiment, the saw blade 760 can further include measurement markings along its length to allow users to precisely set the depth of the saw blade 760.

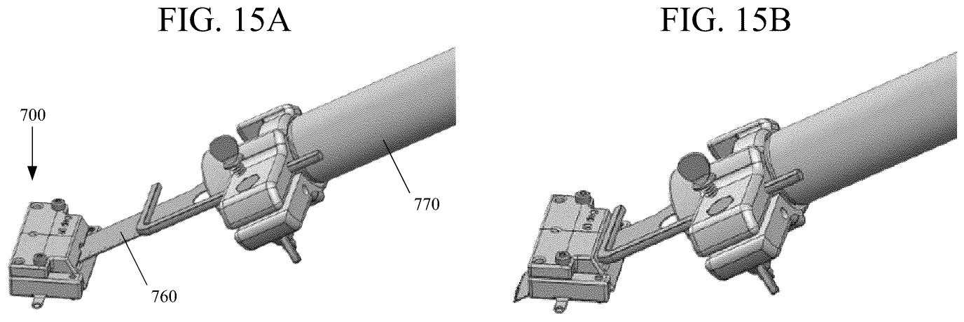

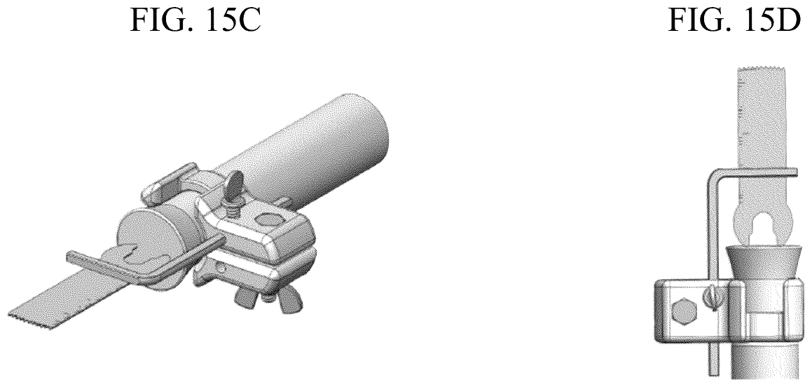

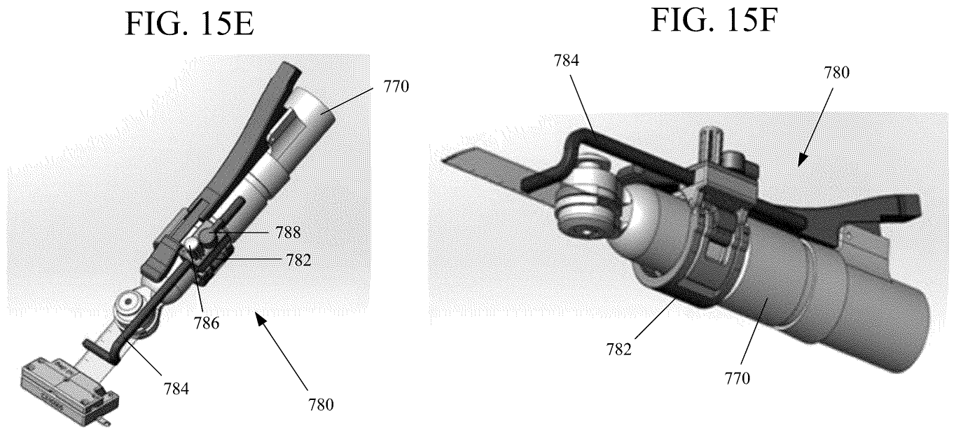

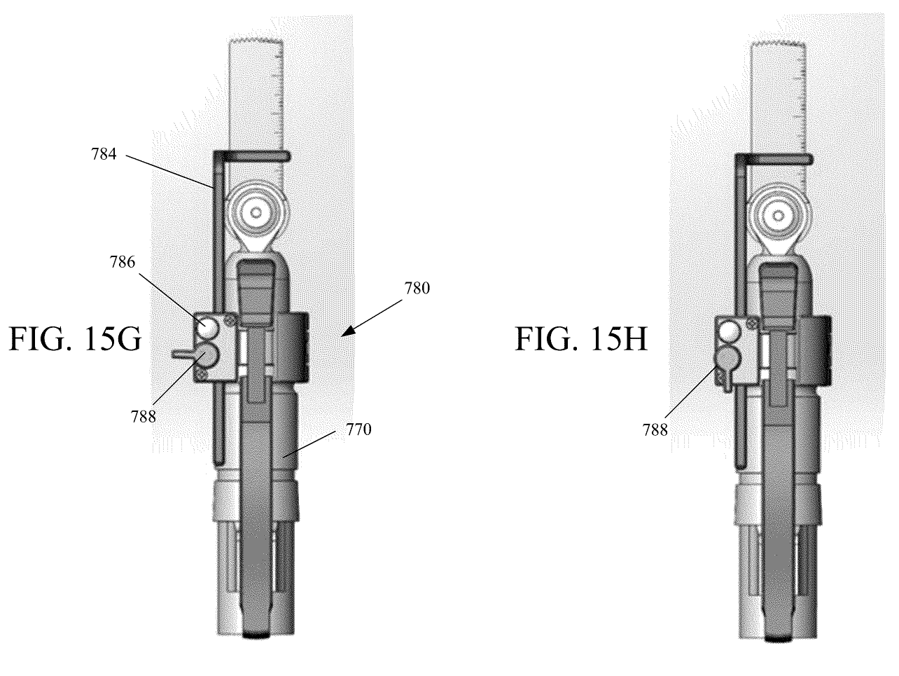

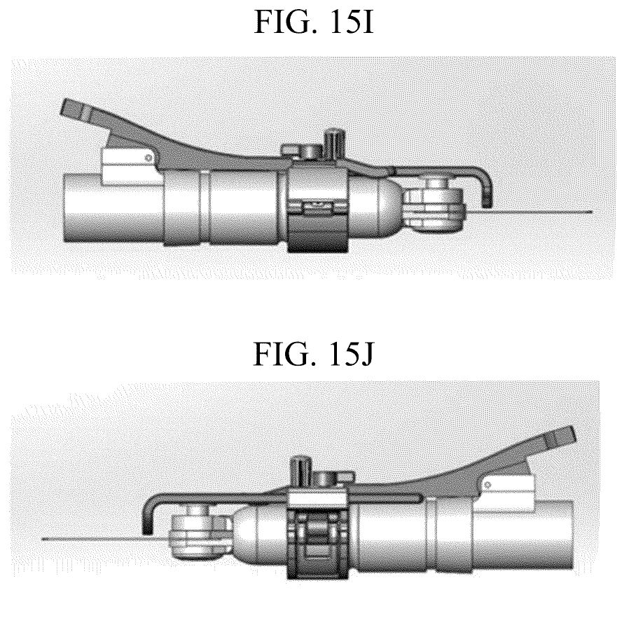

A variety of different saws 770 and/or saw blades 760 can be used with the cranial guide system 700 described herein. For example, the saw 770 can include a sagittal saw handpiece from CONMED®. As another example, the saw blade 760 can include an oscillating saw blade. Further, the saws 770 and saw blades 760 can be secured in a variety of different manners. In one example shown in FIGS. 15A-15D, the saw blades 760 can be secured to the saw handpiece via a clamp assembly.

In some embodiment, the minimally invasive surgical system can further include an adjustable depth stop attachment 780 for a saw 770 (e.g., a sagittal saw), as shown in FIGS. 15E-15J. The adjustable depth stop attachment 780 can include a clamping latch 782 that is configured to affix the adjustable depth stop attachment 780 to the saw 770 and a depth stop rod 784 that limits the cutting depth of the saw when the distal end of the depth stop rod 784 makes contact with the cranial guide system 700, as shown in FIG. 15E. In some embodiments, the adjustable depth stop attachment 780 can further include an adjustment knob 786 for controlling the relative position of the depth stop rod 784. The adjustment knob 786 can allow for fine (e.g., millimeter-scale) adjustment of the position of the depth stop rod 784. In some embodiments, the adjustable depth stop attachment 780 includes a lock 788 (e.g., a cam lock) to secures the depth stop rod 784 in place at the desired depth (e.g., as indicated by depth markings on the saw blade). As shown in FIGS. 15G and 15H, the lock 788 can be transitioned between a locked position (FIG. 15G) where the depth stop rod 784 is fixed in place and an unlocked position (FIG. 15H) where the position of the depth stop rod 784 can be adjusted via the adjustment knob 786. In one embodiment, the adjustable depth stop attachment 780 can be constructed from sterilizable materials.

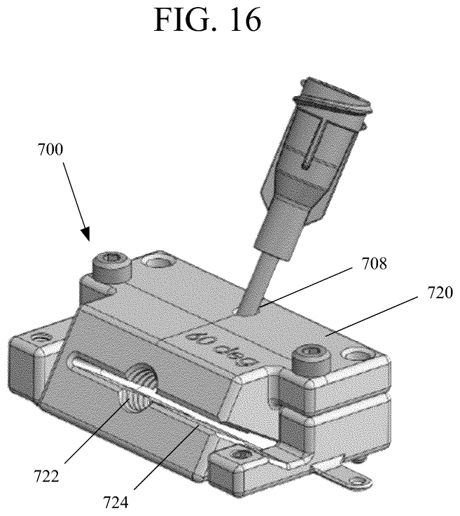

In one embodiment, the cranial guide system 700 can further include an irrigation channel. As one example shown in FIG. 16, the guide portion 720 can include an irrigation channel 708 extending therethrough that is configured to receive an irrigation device and is fluidically coupled to the drill aperture 722 and/or saw slot 724, thereby facilitating irrigation during drilling and/or sawing during the surgical procedure.

### Micro Kerrison Rongeur

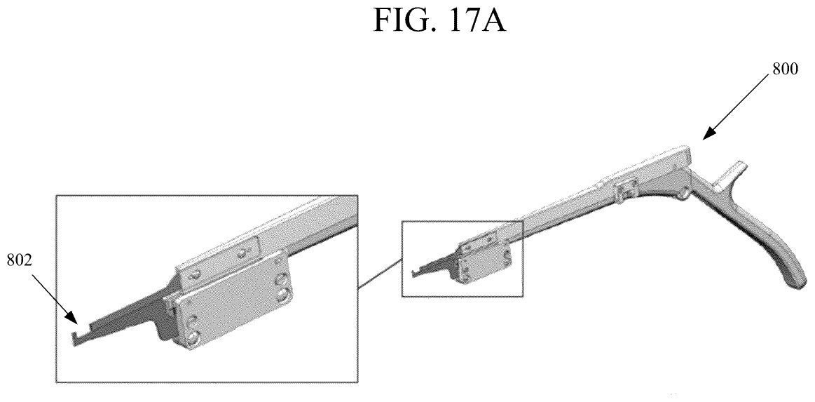

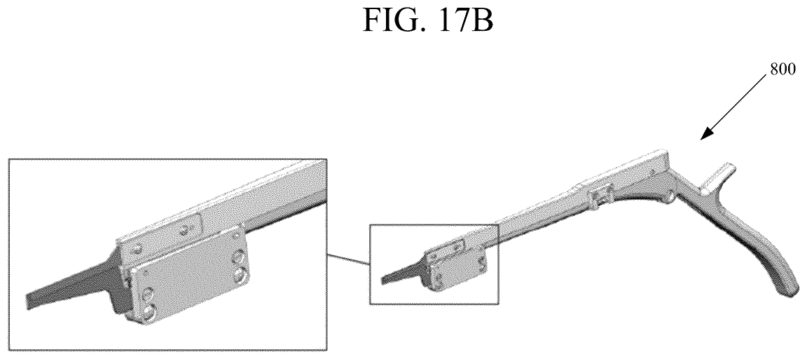

In some embodiments, the surgical processes and techniques described herein can make use of Kerrison rongeurs, which are surgical instruments designed for intraoperatively cutting and removing small pieces of bone. However, conventional Kerrison rongeurs are not dimensioned to access and be used within the confines of the osteotomies created using the surgical techniques and processes described herein. Accordingly, FIGS. 17A and 17B illustrate a micro Kerrison rongeur 800 in an open and closed configuration, respectively. The Kerrison rongeur 800 includes a set of jaws 802 that are dimensioned to operate within the osteotomies created by the surgical procedures described herein. In one embodiment, the Kerrison rongeur 800 can be sized or otherwise configured to be insertable through a surgical slit as small as 0.5 mm. In one embodiment, the Kerrison rongeur 800 can be up to 25 mm in length or otherwise configured to be used down a surgical trajectory as long as 25 mm.

### Stylets, Shims, and Gauges

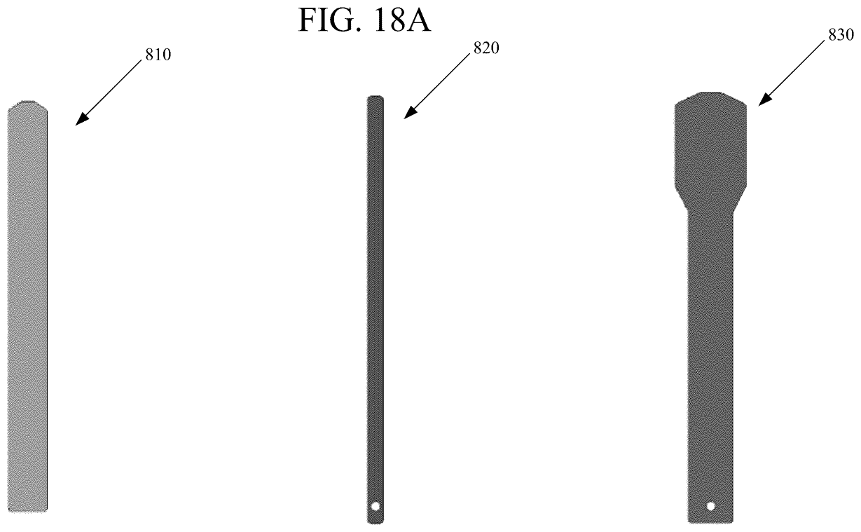

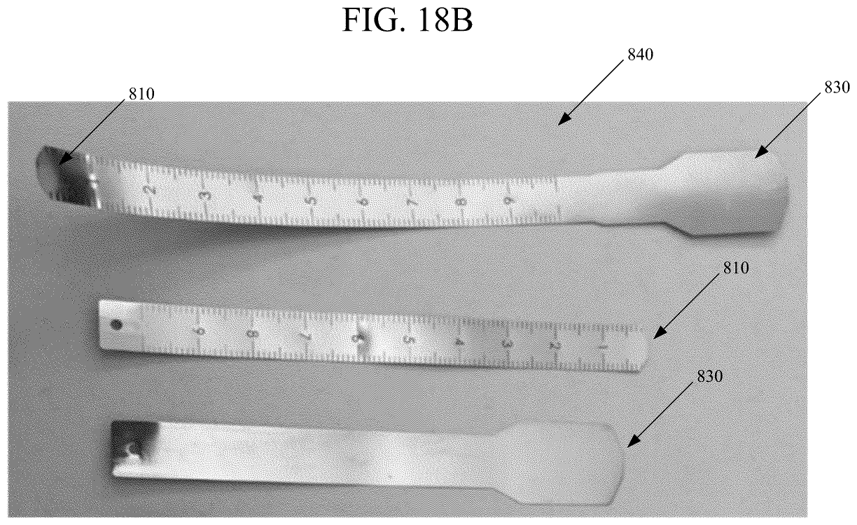

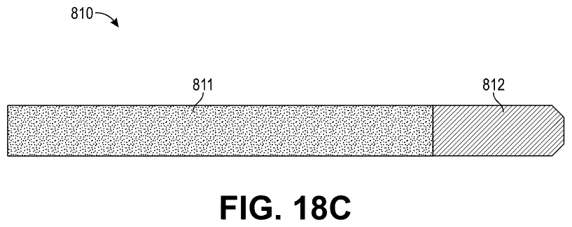

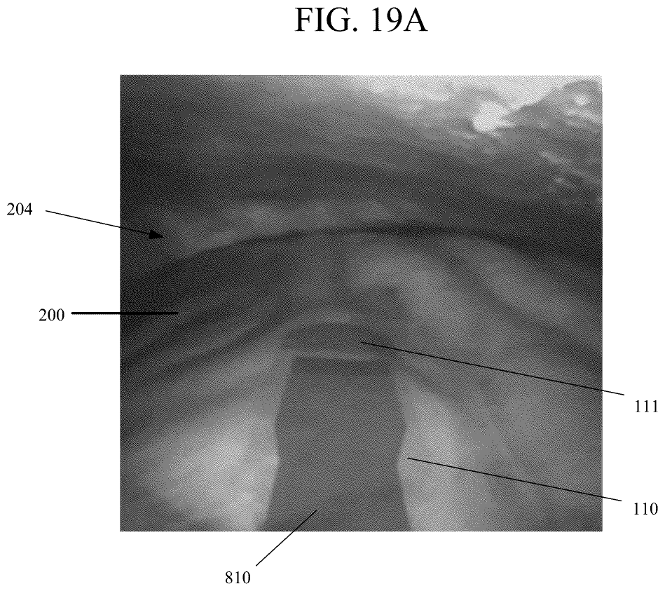

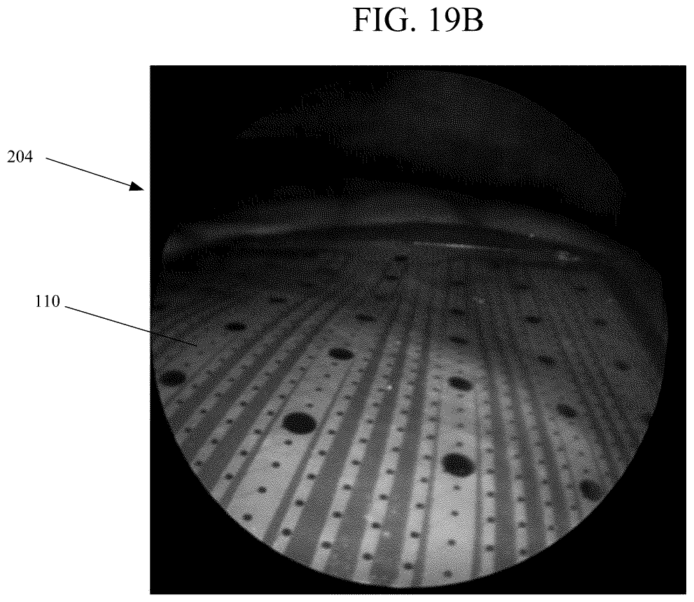

In some embodiments, the surgical processes and techniques described herein can make use of various stylets 810, shims 820, and gauges 830, such as are shown in FIGS. 18A and 18B. The stylet 810 (also referred to as a “deployment stylet”) can be used for the insertion of the neural device 110 through the angular slit 500 to deploy the neural device 110, as described above and shown in FIGS. 19A and 19B. As described above, the neural device 110 can include a pocket 111 (FIG. 19A) that can be sized or otherwise configured to receive the stylet 810. The pocket 111 can be positioned at the distal end of the electrode array 180, for example. In particular, the stylet 810 can engage with the pocket 111 for deploying the electrode array 180 into the subdural space 204. Once the electrode array 180 is positioned against the brain 200, the stylet 810 can be retracted from the pocket 111, while keeping the electrode array 180 in place on the cortical surface. In one embodiment, the stylet 810 can include depth markings for providing visual feedback to the surgeon as to how far the electrode array 180 has been deployed into the subdural space 204.

The shim 820 can provide a variety of different functions in the surgical procedure described herein. For example, the shim 820 can be used to provide haptic feedback to the surgeon in assessing if the inner table of the bone has been drilled fully through. In one embodiment, the shim 820 can include depth markings for providing visual feedback on the depth of the osteotomy. As another example, the shim 820 can be used to help complete the dural incision by pushing through the dura. As yet another example, the shim 820 can be used to clear open the subdural space by gently manipulating any arachnoid layers that may be bridging the dura with the pia mater.

The minimally invasive surgical system described herein can include a variety of different gauges 830. In one embodiment, the gauges 830 can include a width gauge that can be used to ensure the osteotomy and the dural incision are large enough to accommodate the electrode array 180. The width gauge can be sized and shaped to correspond to the electrode array 180.

In some embodiments, the minimally invasive surgical system can include dual or combination tools, i.e., tools that are combinations of the stylets 810, shims 820, and/or gauges 830. For example, the minimally invasive surgical system can include a combination deployment stylet and size gauge tool 840, as shown in FIG. 18B. This combination tool 840 can be a double-ended tool where one end is a deployment stylet 810 (e.g., including the aforementioned depth markings) and the other end is a size gauge 830.

In various embodiments, the stylet 810, shim 820, gauge 830, and/or any combination tool thereof can be coated, partially coated, or electropolished. In embodiments where the stylet 810, shim 820, and/or gauge 830 includes any exposed metal, the exposed metal portion of the stylet 810 can be passivated. In some embodiments, the stylets 810, shims 820, and gauges 830 can include various coatings. The coatings can include various materials that have been approved for use in surgical applications. For example, the coatings can include polytetrafluoroethylene, Xylan® 8110, Eclipse®, plasma polymerized coating, diamond-like carbon (DLC) coating, lubricious silicone coating, and any combination thereof. In various embodiments, the aforementioned tools can be completely or partially coated. For example, FIG. 18C depicts a stylet 810 comprising an uncoated portion 811 and a coated portion 812, wherein the coated portion 812 includes one or more of the coatings described above.

### Visualization

In some embodiments, the surgical processes and techniques described herein can include a step of inserting an endoscope 850 through the angular slit 500 and/or pilot hole 502 to visually access the subdural space and confirm that the angular slit 500 and/or pilot hole 502 have been formed properly. In one embodiment, the minimally invasive surgical system can further include a docking tool 852 for the endoscope 850 that is configured to allow for both rotational and linear/axial movement of the endoscope 850. Accordingly, the docking tool 852 can provide a hands-free view of the interior of the osteotomy. In another embodiment, a conventional docking tool can be utilized for holding the endoscope 850.

In some embodiments, one or more components of the surgical system that are introduced into the subdural space can include fiducials, radiopaque markers that can be visualized under various imaging modalities (e.g., fluoroscopy or CT), or other markers that can be tracked using various tracking techniques (e.g., electromagnetic tracking). In some embodiments, one or more components of the surgical system can be trackable via real-time electrophysiology techniques.

### Micro Picks

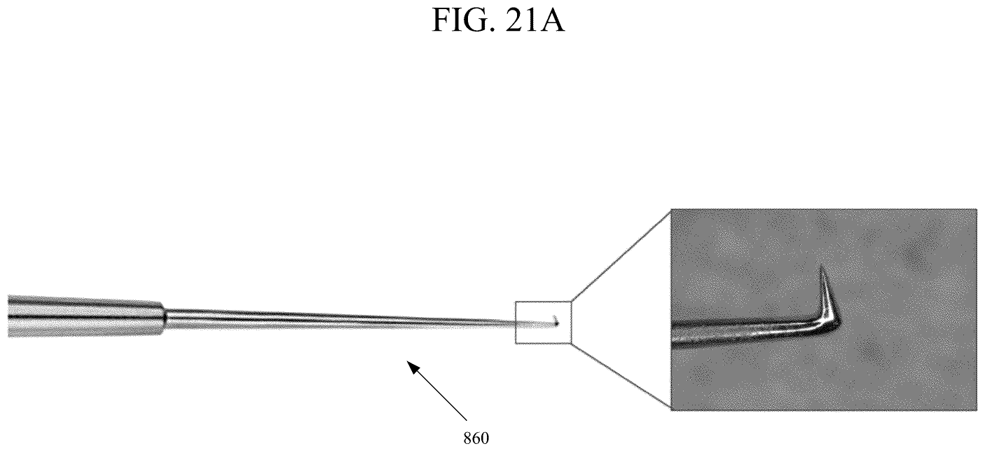

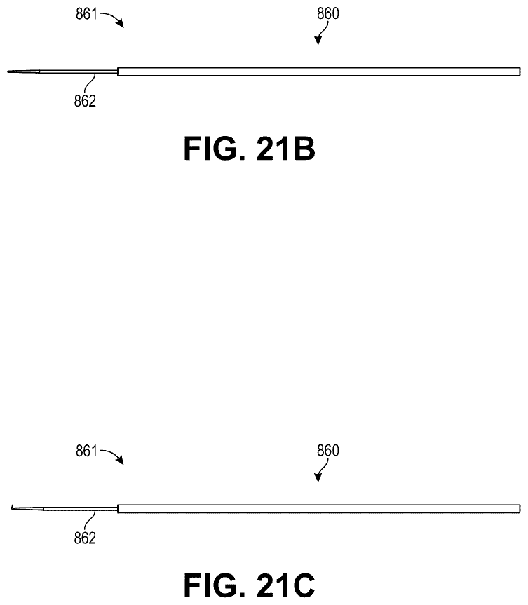

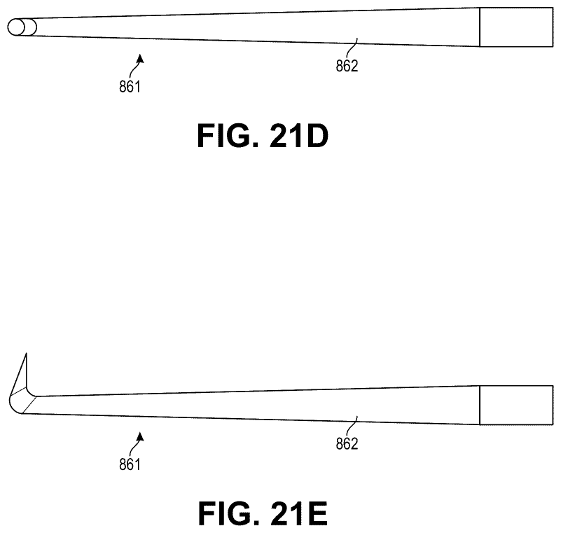

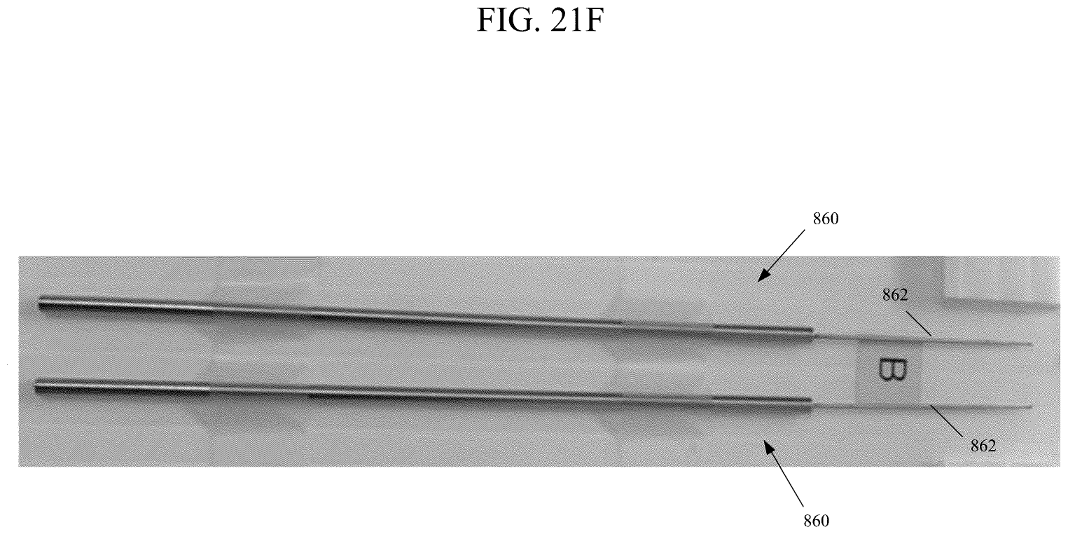

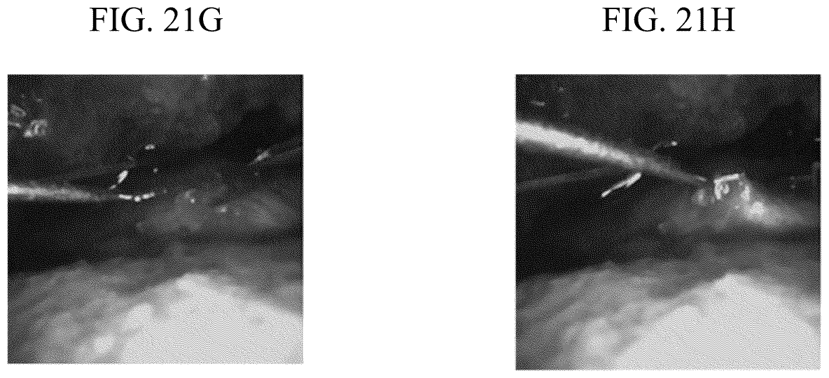

In some embodiments, the surgical processes and techniques described herein can make use of various different types of picks. Similarly as with the Kerrison rongeurs, conventional picks are not dimensioned to access and be used within the confines of the osteotomies created using the surgical techniques and processes described herein. For example, FIGS. 21A-21H illustrate an embodiment of a tissue pick 860 dimensioned to operate within the osteotomies created by the surgical procedures described herein. In particular, FIGS. 21A, 21B, 21C, and 21F illustrate the tissue pick 860; FIGS. 21D and 21E illustrate the distal end 861 of the tissue pick 860; and FIGS. 21G and 21H illustrate the tissue pick 860 in use. In one embodiment, the tissue pick 860 can be configured to work within a 0.3 mm thickness space and down a trajectory length from about 20 mm to about 30 mm. Further, FIGS. 21G and 21H illustrate how the properly dimensioned tissue pick 860 can be inserted through the angular slit 500 and used intraoperatively to lift dura or other tissue, which conventionally sized tissue picks would be unable to do because of their size. In one embodiment, the tissue picks 860 for use with the minimally invasive surgical system described herein can include an extend tapered region 862 that is configured to provide a longer reach into the slit osteotomy.

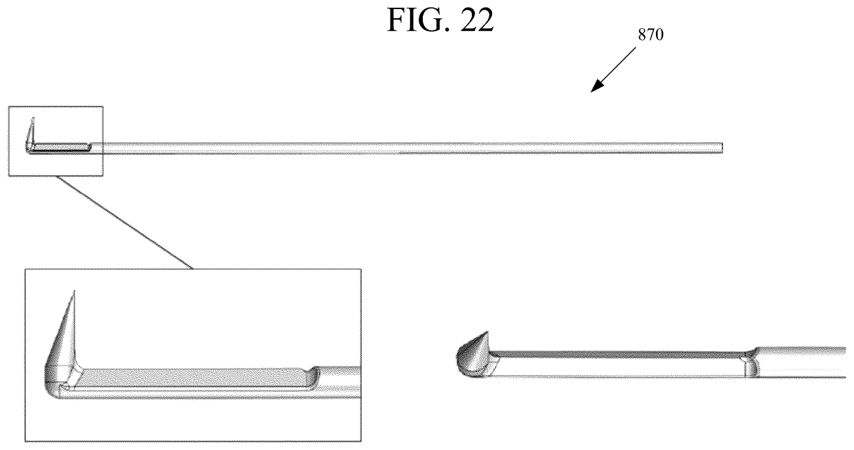

As another example, FIG. 22 illustrates an embodiment of a pick knife 870 dimensioned to operate within the osteotomies created by the surgical procedures described herein. In some embodiments, the pick knife 870 can be dimensioned similarly or the same as the tissue pick 860. In one embodiment, the pick knife 870 can be configured to work within a 0.3 mm thickness space and down a trajectory length from about 20 mm to about 30 mm. In one embodiment, the knife edge of the pick knife 870 can be from about 1 mm to about 1.5 mm long. The pick knife 870 can be inserted through the angular slit 500 and used intraoperatively to lift and/or incise the dura or other tissue, which conventionally sized pick knives picks would be unable to do because of their size.

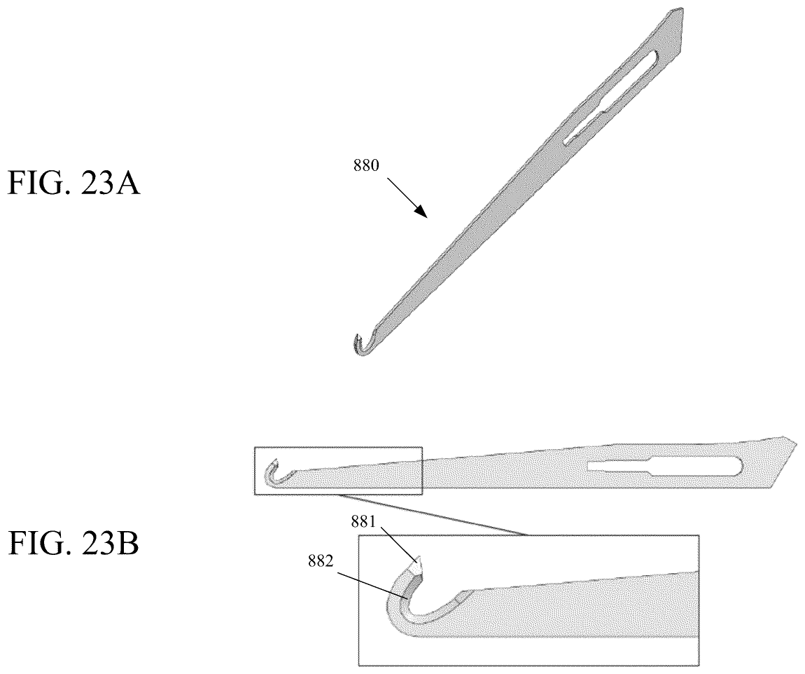

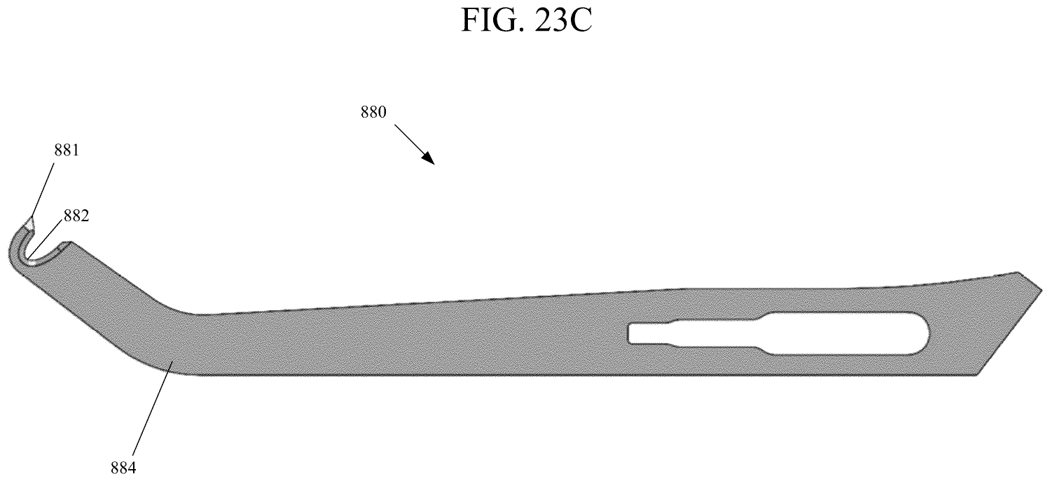

As yet another example, FIGS. 23A and 23B illustrate an embodiment of a hook knife 880 dimensioned to operate within the osteotomies created by the surgical procedures described herein. The hook knife 880 can include a sharp tip 881 configured to penetrate intact dura and a curved cutting edge 882 to catch the dura and cut as the hook knife 880 is pulled along the osteotomy. In one embodiment, the hook knife 880 can be about 0.4 mm thick and 62 mm long. In one embodiment, the hook knife 880 can be configured to work down a trajectory length of up to about 35 mm. In some embodiments, the hook knife 880 can be engaged with a conventional scalpel handle, such as a standard size three scalpel handle. Accordingly, the hook knife 880 can be inserted through the angular slit 500 and used intraoperatively to lift and/or incise the dura or other tissue, which conventionally sized hook knives would be unable to do because of their size. In one embodiment, the hook knife 880 can be substantially linear as shown in FIGS. 23A and 23B. In an alternative embodiment shown in FIG. 23C, the hook knife 880 can further include a bend 884 that causes the cutting edge 882 and tip 881 to be positioned off-center with respect to the longitudinal axis of the body of the hook knife 880. This alternative embodiment of the hook knife 880 can be beneficial in order to allow for improved endoscopic visualization of the tip 881 while in use, for example.

In some embodiments, the various picks 860 and/or knives 870, 880 described herein can include various coatings, as described above. In other embodiments where the picks 860 and/or knives 870, 880 do not include coatings, the bare metal can be passivated (e.g., in accordance with medical device regulatory standards).

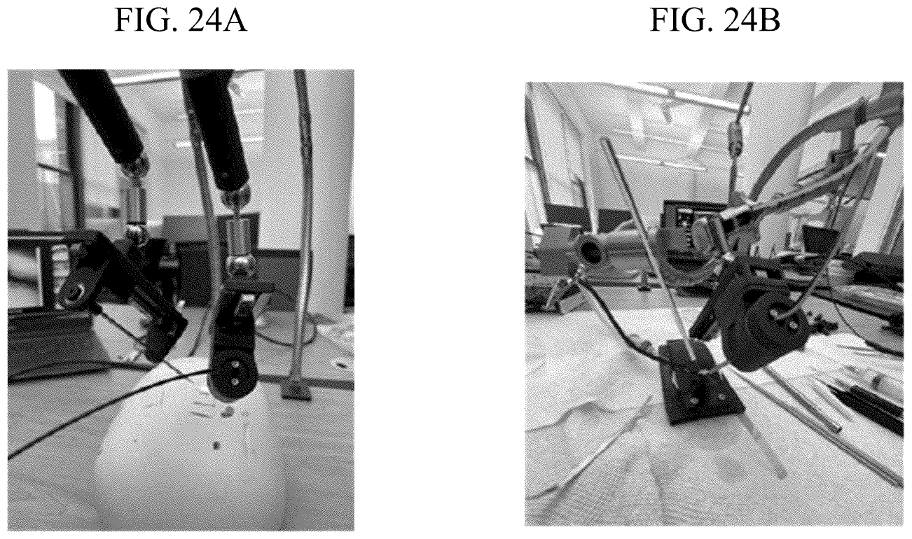

Referring now to FIGS. 24A and 24B, there are shown views of a surgical setup. In particular, the aforementioned dura picking and/or incising tools can be intraoperatively docked alongside the endoscope docking tool, thereby allowing the surgical team to intraoperatively visualize the use of the surgical tools.

### Neural Interface Anchors

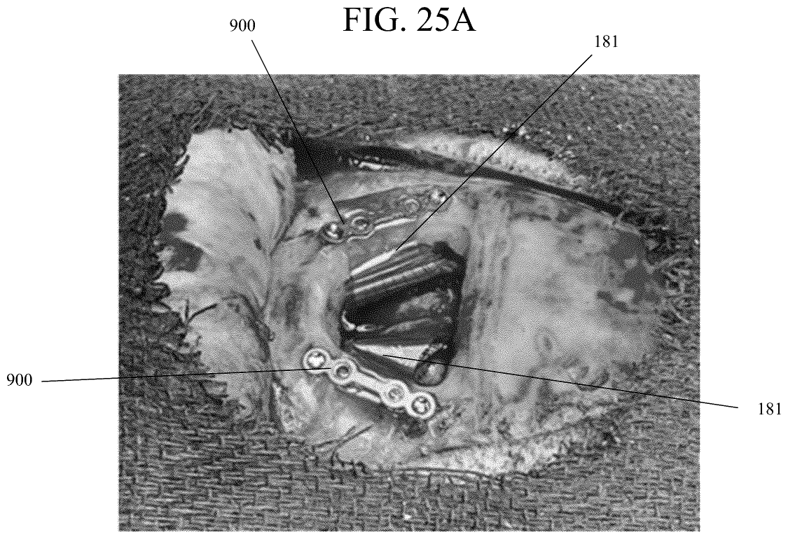

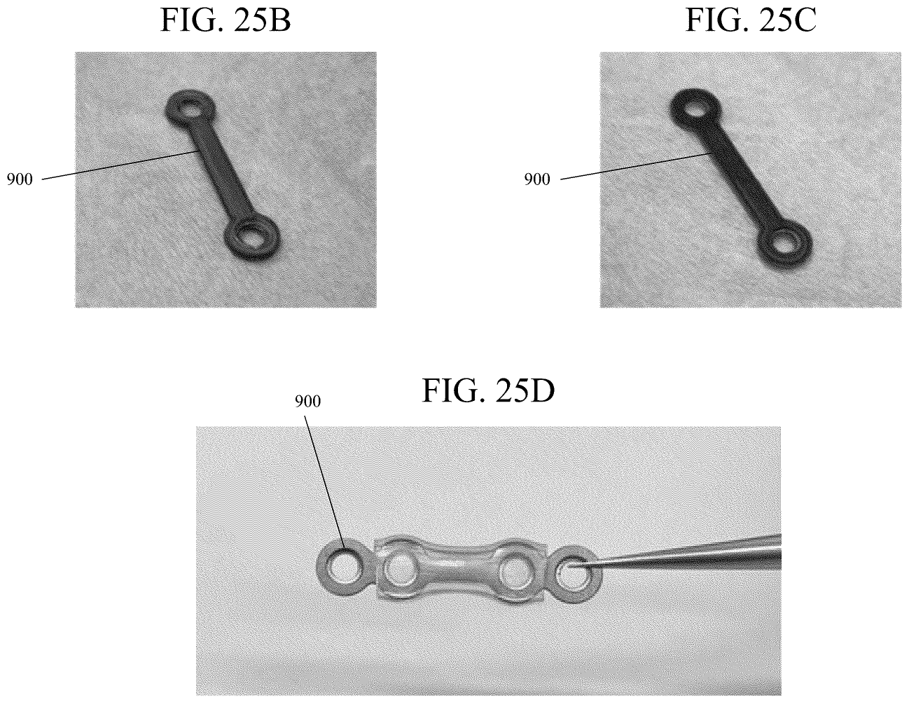

As described above, the minimally invasive surgical techniques described herein involve cutting an osteotomy and then inserting the electrode array 180 therethrough to deploy the electrode array 180 into the subdural space against the cortical surface. In some embodiments, the minimally invasive surgical system can include anchors 900 for securing the electrode array cables 181 in place once the electrode array 180 has been surgically deployed, as shown in FIG. 25A. The anchors 900 can be constructed from a variety of different materials, including silicone, titanium, or a combination thereof. The anchors 900 can be configured to be compatible with a variety of different surgical fasteners, such as standard 1.9 mm diameter bone screws. In one embodiment shown in FIGS. 25B and 25C, the anchors 900 can be color coordinated to differentiate between a first side (visible in FIG. 25B), which is intended to be oriented away from the patient, and a second side (visible in FIG. 25C), which is intended to contact the patient. The embodiment shown in FIGS. 25B and 25C is constructed from overmolded silicone. In another embodiment shown in FIG. 25D, the array anchors 900 can be constructed from a material (e.g., titanium) and sheathed with a silicone tube. The silicone tubing can be about 12 mm to about 13 mm long.

This disclosure is not limited to the particular systems, devices, and methods described, as these may vary. The terminology used in the description is for the purpose of describing the particular versions or embodiments only and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the term “implantable medical device” includes any device that is at least partially introduced, either surgically or medically, into the body of a subject and is intended to remain there after the procedure.

As used herein, the singular forms “a,” “an,” and “the” include plural references, unless the context clearly dictates otherwise. Thus, for example, reference to a “protein” is a reference to one or more proteins and equivalents thereof known to those skilled in the art, and so forth.

As used herein, the term “about” means plus or minus 10% of the numerical value of the number with which it is being used. Therefore, about 50 mm means in the range of 45 mm to 55 mm.

As used herein, the term “consists of” or “consisting of” means that the device or method includes only the elements, steps, or ingredients specifically recited in the particular claimed embodiment or claim.

In embodiments or claims where the term “comprising” is used as the transition phrase, such embodiments can also be envisioned with replacement of the term “comprising” with the terms “consisting of” or “consisting essentially of.”

As used herein, the term “subject” includes, but is not limited to, humans and non-human vertebrates such as wild, domestic, and farm animals.

While the present disclosure has been illustrated by the description of exemplary embodiments thereof, and while the embodiments have been described in certain detail, it is not the intention of the Applicants to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the Applicant's general inventive concept.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A cranial guide for surgically preparing a subject for an implantable neural device, the cranial guide comprising:
a saw slot for receiving an oscillating saw blade, the saw slot oriented to form an angular slit in the subject oriented from about 45° to about 65°; and
a drill aperture for receiving a drill bit therethrough, the drill aperture coextensive with the saw slot.

2. The cranial guide of claim 1, further comprising leveling pins to adjust a relative position or orientation of the cranial guide relative to the subject.

3. The cranial guide of claim 1, further comprising a friction-reducing coating disposed on an inner surface of the saw slot.

4. The cranial guide of claim 3, wherein the coating comprises at least one of polytetrafluoroethylene, Xylan® 8110, Eclipse®, plasma polymerized coating, diamond-like carbon (DLC) coating, or lubricious silicone coating.

5. The cranial guide of claim 1, wherein the drill aperture is disposed at a midpoint of the saw slot.

6. The cranial guide of claim 1, wherein a thickness of the saw slot is from about 0.4 mm to about 0.8 mm.

7. The cranial guide of claim 1, wherein a thickness of the saw slot is from about 0.8 mm to about 1 mm.

8. The cranial guide of claim 1, wherein a width of the drill aperture is from about 0.5 mm to about 3 mm.

9. A method for surgically preparing a subject for an implantable neural device, the method comprising:
placing an incision template on a head of the subject, the incision template comprising a first portion and a second portion arranged orthogonally with respect to the first portion, the first portion defining a slit configured to receive a cutting instrument therethrough, wherein a first length of the slit corresponds to a second length of an angular slit to be formed in the subject;
using the incision template, making an incision in a scalp of the subject with the cutting instrument;
placing a cranial guide on the scalp of the subject at the incision, the cranial guide comprising a saw slot for receiving an oscillating saw blade and a drill aperture for receiving a drill bit therethrough, wherein the drill aperture is coextensive with the saw slot;
using the drill aperture of the cranial guide, drilling a pilot hole through the scalp and into a skull of the subject; and
using the saw slot, cutting the angular slit through the scalp and into the skull of the subject, wherein oriented from about 45° to about 65°.

10. The method of claim 9, wherein the cranial guide further comprises leveling pins, wherein the method further comprising adjusting, via the leveling pins, a relative position or orientation of the cranial guide relative to the subject such that rotation or tilt of the cranial guide is prevented.

11. The method of claim 9, wherein the cranial guide further comprises a friction-reducing coating disposed on an inner surface of the saw slot.

12. The method of claim 11, wherein the coating comprises at least one of polytetrafluoroethylene, Xylan® 8110, Eclipse®, plasma polymerized coating, diamond-like carbon (DLC) coating, or lubricious silicone coating.

13. The method of claim 9, wherein the drill aperture is disposed at a midpoint of the saw slot.

14. The method of claim 9, wherein a thickness of the saw slot is from about 0.4 mm to about 0.8 mm.

15. The method of claim 9, wherein a thickness of the saw slot is from about 0.8 mm to about 1 mm.

16. The method of claim 9, wherein a width of the drill aperture is from about 0.5 mm to about 3 mm.
